(19) United States
(12) Patent Application Publication
(10) Pub. No.: US 2001/0024796 A1
(43) Pub. Date: Sep. 27, 2001

(54) Methods for parallel detection of compositions having desired characteristics

(76) Inventors: Sergey A. Selifonov, Gainesville, FL (US); Gjalt W. Huisman, San Carlos, CA (US)

Correspondence Address: LAW OFFICES OF JONATHAN ALAN QUINE, P O BOX 458, ALAMEDA, CA, 94501

(21) Appl. No.: 09/738,544

(22) Filed: Dec. 15, 2000

Related U.S. Application Data
(60) Provisional application No. 60/172,394, filed on Dec. 17, 1999.
(60) Provisional application No. 60/220,921, filed on Jul. 26, 2000.

(51) Int. Cl. G01N 33/53

(52) U.S. Cl. 435/7.1; 324/308

(57) ABSTRACT

Methods and apparatuses for performing high throughput magnetic resonance imaging spectroscopy, e.g., to screen libraries of chemical or biological compositions for a compound of interest, are provided. Methods of identifying metabolic disorder genes, modulatory compounds and catalysts, and methods of optimizing reaction conditions, using high throughout MRI screening, are also provided.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### CROSS-REFERENCE TO RELATED APPLICATIONS

The present application claims benefit of and priority to U.S. Ser. No. 60/172,394, “Methods for Parallel Detection of Compositions Having Desired Characteristics,” by Selifonov and Huisman, filed Dec. 17, 1999; U.S. Ser. No. 60/220,921 entitled “Methods for Parallel Detection of Compositions Having Desired Characteristics,” by Selifonov and Huisman, filed Jul. 26, 2000; and co-filed PCT application, “Methods for Parallel Detection of Compositions Having Desired Characteristics,” by Selifonov and Huisman, filed Dec. 15, 2000, Attorney Docket No. 02-101220PC.

### BACKGROUND OF THE INVENTION

Reproducible analytical methods that can be applied in a high throughput fashion for detection of variants with improved or desired properties are desired, e.g., in the application of various directed evolution technologies, e.g., DNA shuffling, for improvement of properties of objects, e.g., enzymes, metabolic pathways, organisms, and the like. In many cases, especially when dealing with various biocatalytic chemical processes, a need exists for uniform, sensitive, and fast analysis of variations in chemical compositions that are affected by changes in the genes obtained by the above genomic methods. While many methods exist in the art, including various mass-spectral techniques, UV-VIS spectral methods, chromatography, and radioassays, many of the compounds of interest cannot be conveniently analyzed by these methods due to various reasons, such as complex and labor consuming sample work-up and derivatization procedures, potential hazards, lack of reproducibility, analyte interference, and need for specialty substrates, complex syntheses, or radiotracers.

One well established and mature technique widely used to provide non-invasive medicinal diagnostic and research applications is magnetic resonance imaging spectroscopy. Magnetic resonance imaging (MRI) techniques are typically used for metabolic mapping of large biological objects, such as whole mammalian organisms, body parts, and organs. This technique is based on nuclear magnetic resonance spectroscopy, which is typically used for sample analysis and identification in chemistry. However, this technique is not typically used in a high throughput manner.

However high throughput MRI imaging techniques capable of screening a plurality of biological compositions, such as the activity of a library of shuffled genes would be useful. The present invention fulfills these and other needs that will become apparent upon complete review of this disclosure.

### SUMMARY OF THE INVENTION

The present invention relates to the surprising discovery that up to thousands of samples, e.g., cells, biological compositions, chemical reaction compositions, and the like, can be simultaneously assessed for the presence and/or quantity of metabolic compounds, cofactors, and chemical reaction products, as well as various ions, metals, side products and the like, using magnetic resonance imaging (NRI). To simultaneously perform MRI on a plurality of samples, a physical array comprising a plurality of sample compartments is placed within the magnetic field and analyzed. Imaging techniques are then used to correlate spectroscopic signals to a particular sample compartment, thus providing an assessment of each sample in the array. Thus the present invention provides methods and apparatuses for screening a plurality of samples. Applications of these screening methods include, but are not limited to, methods of identifying metabolic disorder genes and methods of identifying modulatory compounds for use in drug development.

In one aspect, a method of screening a plurality of samples for a selected property is provided. The method comprises providing an artificially generated physical array comprising one or more samples at each of a plurality of spatial locations. The physical array is placed in a magnetic field or a magnetic field is applied to the physical array. The magnetic field typically comprises a magnetic field strength of about 1.5 Tesla or more. MRI spectroscopy is then performed on the one or more samples within the physical array, thereby identifying a spatial location for each of the one or more samples. The samples are identified by one or more MRI detectable chemical shifts that correspond to the selected property. The plurality of samples is thereby screened for the selected property.

Performing MRI spectroscopy in the present invention typically comprises exciting atomic nuclei in the samples, thereby producing a plurality of signals. The signals are detected and images are generated from the signals. In one embodiment, the images are obtained by applying spiral-based k-space trajectories. The images generated correspond to the plurality of spatial locations. The images are analyzed for the presence of one or more selected chemical shifts that correspond to the selected property. The images and chemical shifts are then deconvoluted to provide the spatial location for each of the one or more samples having the selected property. Therefore, the plurality of samples is simultaneously screened for the selected property.

Typically, one or more selected chemical shifts are measured for each of the one or more samples simultaneously. At least about 3000 to about 50,000 samples are optionally screened per hour using the methods of the present invention.

The samples to be analyzed by the methods of the present invention are typically held in a physical array, e.g., a uniform physical array. The array is typically an artificially generated array that comprises one or more spatially separated sample compartments. The sample compartments are optionally positioned in one plane or in multiple planes. The geometric arrangement of the array includes, but is not limited to, a cylindrical array, a square array, a cubical array, a rectangular array, or the like. Alternatively, the array is positioned within a cylindrical structure, a square structure, a cubical structure, a rectangular structure, or the like for analysis in an MRI spectrometer. For example, the array or its structure optionally has a diameter of about 20 cm and a length of about 24 cm. In addition, the array optionally comprises one or more elements that reduce the heterogeneity of a magnetic field, such as water, alcohol, or lipid-based compositions, including, but not limited to, paraffin-filled outer walls, water-filled outer walls, or is surrounded with water.

In one embodiment, the array comprises one or more microwell plates or microtiter plates, e.g., 24-well plates, 96-well plates, 384-well plates, or 1536-well plates. For example, an array comprising multiple microwell plates, e.g., stacked microwell plates, optionally comprises at least about, e.g., 24, 48, 72, 96, 192, 288, 384, 768, 1152, 1536, 3072, 4068, or 6144 different spatial locations.

The number of samples that can be contained in a physical array of the invention ranges from at least about 12 to about 10,000 samples. For example, the physical arrays of the invention optionally comprise at least about 24, 48, 72, 96, 192, 288, 384, 768, 1152, 1536, 3072, 4068, or 6144 or more samples.

The samples analyzed using the apparatus and methods of the present invention include, but are not limited to, a library of biological or chemical compositions, such as a library of expression products or variant genes or a library of mutagenized cells. Such libraries are optionally generated by DNA shuffling, random mutagenesis, transposon mutagenesis, or combinatorial gene assembly. Gene libraries are optionally expressed to produce libraries of expression products. Alternatively, the plurality of samples comprises one or more of: a microbial cell culture, a mammalian cell culture, a cell biomass, a culture broth, an extract, a reaction mixture, a plant tissue sample, a fruit sample, a root sample, a tuber sample, and a plant seed.

Typically, the samples of the invention contain at least one MRI active compound. Such compounds typically comprise ¹H, ¹³C, ¹⁵N, 33S, ³¹P, or ¹⁹F. Chiral shift reagents, paramagnetic ions, and/or ferromagnetic ions are also optionally included in the samples of the invention. For example, to measure metal uptake ferromagnetic ions are optionally added to the samples.

The samples and/or the sample compartments or wells of the invention typically have a standard volume, a standard size, and a standard geometry. For example, the standard volume typically ranges from about 1 μl to about 20 ml, preferably from about 100 μl to about 10 ml, and more preferably from about 1.5 ml to about 2.0 ml.

The samples of the invention are typically assessed for a selected property, such as metal uptake level, a selected pH, or the like. To measure a selected pH in the plurality of samples, MRI is used to measure a phenolic proton signal corresponding to each sample. Alternatively, the selected property is a selected amount of a compound of interest or the presence of a compound of interest in the samples. In some embodiments, the plurality of samples comprises one or more microbial strains expressing a library of shuffled genes or a genomic library. Performing MRI on these samples provides a comparison of expression levels for each member of the library of shuffled genes or of the genomic library. In other embodiments, MRI spectroscopy provides a comparison of the performance of a selected biological composition under two or more different conditions.

Compounds of interest that are detected and/or measured by MRI include, but are not limited to, hydroxy acids, alcohols, polyols, carboxylic acids, lactones, esters, polyhydroxyalkanoates, terpenoids, carotenoids, steroids, polyketides, lipids, triglycerides, aromatics, amino acids, alkenes, vitamins, halogenated organic compounds, benzene bioconversion products, toluene bioconversion products, ethylbenzene bioconversion products, xylene bioconversion products, monosaccharides, and polysaccharides. For example, the compound of interest is optionally lactate, citrate, tylosin, 1,3-propanediol, ethanol, succinate, glycerol, itaconate, PHB/PHA, lysine, threonine, isoleucine, methionine, tryptophan, phenylalanine, tyrosine, glycine, valine, glutamate, aspartate, histidine, phytohaemagglutinin-A, phytohaemagglutinin-B, p-hydroxybenzoate, 3-hydroxybutyrate, aspartame, N-methylaspartate, ε-caprolactone, or the like. For example, performing MRI spectroscopy optionally provides a comparison of the absolute or relative amount, e.g., of any of the above compounds, in each sample of the physical array.

An apparatus for screening a plurality of samples is also provided in the present invention. An apparatus of the invention comprises a magnetic resonance imaging spectrometer; and at least one microwell plate or other artificially generated physical array, as described above. During operation of the apparatus, the at least one microwell plate or other artificially generated physical array is positioned within a magnetic field produced by the magnetic resonance imaging spectrometer. The magnetic field typically comprises a field strength of about 1.5 Tesla or more. The plurality of samples is placed into the physical array and the physical array is placed within the magnetic field of the MRI spectrometer for analysis as described above. Using the methods of the invention, at least about 3000 to about 50,000 samples or more are optionally screened per hour. Alternatively, samples are optionally screened at a rate of about 1×10⁶ samples per day.

In other embodiments, the apparatus comprises an automatic sampler operably coupled to the MRI spectrometer. The automatic sampler positions the microwell plate(s) or other artificially generated physical array within the magnetic resonance imaging spectrometer.

In another embodiment, the apparatus further comprises a detector operably coupled to the spectrometer. The detector detects signals generated by operation of the MRI spectrometer. A computer and software are optionally coupled to the apparatus for recording and analyzing data from the MRI spectrometer.

In another aspect, the present invention provides MRI screening applications. In one embodiment, a method of identifying metabolic disorder genes is provided. Metabolic disorders of interest include, but are not limited to, colon cancer, batten disease, deafness distonia syndrome, and lupus nephritis. The method comprises providing a plurality of cells. Typically, the cells have been transformed with a plasmid containing one or more members of a library of gene sequences, e.g., gene sequences corresponding to a metabolic disorder mutants, and produce one or more metabolites. Alternatively, the plurality of cells represents a mutagenized collection of a cell of interest. Alternatively, the plurality of cells comprises derivatives of the cells, such as cellular extracts; other biological samples (including, but not limited to, blood, serum; saliva, sputum, tears, sweat, cerebrospinal fluid, interstitial fluid, and other biologically-derived fluids), biologically-altered solutions preciously exposed to the plurality of cells (such as tissue culture medium or bioreactor medium), and the like. The cells are screened by performing MRI spectroscopy, using physical arrays as described above, on the plurality of cells or on the metabolites. The screening is used to detect the metabolites and identify cells that produce a reduced or increased level of at least one of the metabolites. One or more metabolic disorder genes are identified by identifying the gene sequence in those cells with reduced or increased levels of metabolites.

In another embodiment, a method of identifying a modulatory compound is provided. The method comprises providing a plurality of cells and screening them as described above for cells and/or gene sequences that yield a reduced or increased level of at least one metabolite. These genes are optionally shuffled to determine interdependence between the various gene sequences of the library. Alternatively, the gene or genes identified are used to create a library of shuffled genes that is reintroduced into a host and screened, e.g., by MRI, for genes with improved properties.

Once cells and/or gene sequences with reduced or increased metabolite production are identified, potential modulatory compounds are screened for alleviation of the reduced or increased level of metabolites by performing MRI spectroscopy as described above. The cells are incubated with one or more potential modulatory compounds. MRI spectroscopy is performed on the cells and/or metabolites produced in the presence of the potential modulatory compounds. Metabolite production levels for the cells with and without the potential modulators are compared and/or quantified, thus identifying modulatory compounds that alleviate the reduced or increased metabolite level.

Potential modulatory compounds of the present invention include, but are not limited to, peptides, proteins, metabolic products, carbohydrates, lipids, nucleic acids, nucleotides, oligonucleotides, small organic molecules, and combinations thereof.

Cells used to identify metabolic disorder genes and/or modulatory compounds include, but are not limited to, yeast cells, bacterial cells, plant cells, tissue cultures, primary cells, e.g., blood, lymph, tissue, and the like, callus cultures, insect cells, germinating seeds, hatching egg cells, and developing embryo cells, and the like. For example a typical cell comprises a Saccharomyces cerevisiae cell that has been transformed with a library of gene sequences, e.g., a library of shuffled genes in a particular host. Alternatively, a library of mutant cells, e.g., derived from the same parent is studied, e.g., to identify a mutant for a specific gene. The metabolic production of a mutant yeast cell is typically compared with the metabolite production in a standard or non-mutant cell.

The metabolites produced by the cells are typically the same types of compounds as the compounds of interest listed above. For example, ethanol and lactate are typical metabolites that can be measured by MRI according to the present invention.

Using the MRI methods of the present invention, as described above, an entire library of genes, e.g., a 1000-1,000,000 member library, is optionally screened, e.g., to identify metabolic disorder genes, in about 1 hour. Likewise, an entire library of potential modulatory compounds, whole organisms, or mutated cells is simultaneously screened in about an hour.

These and other novel features, advantages, and embodiments of the present invention will become apparent after consideration of the following detailed description of the invention

### BRIEF DESCRIPTION OF THE FIGURES

FIG. 1 provides a schematic illustration of an apparatus of the invention, comprising an MRI spectrometer and an artificially generated physical array.

FIG. 2 shows an MRI signal from four samples of 30 mM glucose. The four samples were positioned in four different wells of a microtiter dish, which served as the physical array, and were scanned simultaneously.

FIGS. 3-6 show MRI images (photographic screen capture data) for a physical array comprising twelve 96-well microwell plates in which the wells contain different amounts of ethanol in water. All 12 microwell plates were simultaneously screened using a method and apparatus of the invention. The panels represent images obtained from MRI spectroscopy on a physical array. Each rectangular grouping corresponds to one of the microwell plates in the array.

FIG. 3: Panel A shows an image of a water spectrum for the top two layers of the array, i.e., the top two microwell plates. Panel B shows an image of an ethanol spectrum for the same microwell plates. Panel C provides a field map indicative of the magnetic filed homogeneity over the space of the two microwell plates. Panel D provides a key to the percent by volume of ethanol in each well of the array.

FIG. 4 shows another layer of the physical array described in FIG. 3. This layer comprised four individual 96-well microwell plates. Panel A provides an image of the water spectrum, Panel B provides an image of the ethanol spectrum, Panel C is the field map and Panel D is a key to the concentrations of ethanol.

FIG. 5 provides the third layer of the array, which comprises four 96-well microwell plates. Panel A provides a water spectrum, Panel B an ethanol spectrum, Panel C a field map, and Panel D a key to ethanol concentrations.

FIG. 6 shows the fourth layer of the physical array, which comprised two 96-well plates. Panel A provides a water spectrum, Panel B an ethanol spectrum, Panel C, a field map, and Panel D a concentration key.

FIG. 7, Panels A through D, provide alternative images of the same microwell plate array used in FIGS. 3-6. Panel E shows a spectrum of the ethanol in one well of the array and Panel F provides a schematic of the array and indicates the direction of imaged slices used in Panels A-D.

FIG. 8 provides a sample array of the invention. Panel A is a schematic representation of a 96-well microtiter plate. Panel B provides front and side views of an array containing twelve 96-well plates, 1-12. Panel C provides an image of the array within the MRI imager.

FIG. 9 provides a quantitative representation, in terms of brightness vs. concentration, of ethanol samples from Plate 11, as depicted in FIG. 6.

FIG. 10 provides relative chemical shift positions for a variety of MRI-detectable moieties, and typical fermentation levels for a variety of compounds.

### DETAILED DISCUSSION OF THE INVENTION

The present invention provides methods of performing high throughput magnetic resonance imaging spectroscopy on two-dimensional and three-dimensional physical arrays of multiple, spatially separated vessels and samples, e.g., for simultaneous comparative measurements of compounds of interest. The techniques provide fast and reproducible non-invasive comparison of absolute and relative amounts of compounds of interest in multiple samples, e.g., of biological origin, so that samples having desired chemical compositions are easily detected.

The methods of the invention typically comprise using traditional MRI imaging techniques as described in the art, but applying the techniques to an artificially generated physical array instead of a large biological object, such as a human body. In traditional MRI, a picture of the subject is provided. In the present invention, a picture of the physical array is provided. In traditional MRI, human body images produced correspond to the amount, e.g., of water or fat at precise locations in the human body. In the present invention, the images produced correspond to the amount of a particular compound of interest, e.g., ethanol, lactate or the like, at defined locations in the physical array, e.g., providing the concentration of ethanol in each well of a microwell plate.

The compounds of interest are optionally present in or produced by variants of biological objects and compositions, such as microbial cell cultures, cell biomass, culture broths, reaction mixtures, and the like. Alternatively, the compounds of interest are libraries of compounds, such as libraries of biologically-active compounds, (e.g., insecticidal, herbicidal, biocidal, fungicidal, stimulatory, repressive, and/or pharmaceutical compound libraries), or chemical catalyst libraries. MRI imaging of arrays of multiple samples is particularly useful as a screening method for comparison of biological compositions obtained by expression of libraries of variant genes, such as generated by DNA shuffling, random or transposon mutagenesis, combinatorial gene assembly, or for comparison of various microbial isolates for ability to generate and accumulate compounds of interest.

“Screening,” in the present invention refers to a method of examining a number of compositions, compounds, or samples, e.g., a library of compositions, such as cells, genes, proteins, or the like for a specified desired property, e.g., metal uptake level, pH, level of expression, and the like. A large number of compositions are optionally screened at once in the present invention, e.g., about 10 to about 50,000 compounds or samples. For example, a physical array comprising multiple stacked microwell plates or test tube racks comprises, e.g., about 24, 48, 72, 96, 192, 288, 384, 768, 1152, 1536, 3072, 4068, or 6144 or more sample compartments and/or samples. These compositions are optionally screened simultaneously in a magnetic resonance imaging (MRI) spectrometer, e.g., to determine which of the compounds have a specified pH. In the present invention, the screening is optionally used to determine which compounds in a library possess a desired trait or property, e.g., a specified pH or metal uptake percentage. Optionally, nucleic acids encoding the compounds with the desired property are recombined to create new compounds that possess the desired property in an improved form compared to the original compounds, e.g., a higher or lower level of metal uptake or greater alleviation of disease symptoms.

I. Performing Magnetic Resonance Imaging Spectroscopy

Magnetic resonance imaging (MRI) typically refers to a non-invasive diagnostic technique that produces computerized images of internal body tissues and is based on nuclear magnetic resonance of atoms within the body induced by the application of radio frequency (rf) pulses. In diagnostic MRI spectroscopy, signals from pulsed radio frequency excitation of samples, e.g., solid or semi-solid samples, are subjected to Fourier transformation and converted to three-dimensional images of the interior of the samples. Samples typically comprise, e.g., a brain, heart, knee, back, or the like. The present invention uses this technique to provide an image of a two-dimensional or three-dimensional artificially generated array, e.g., a microwell or microtiter plate, comprising a plurality of samples, e.g., liquid samples, such as a library of biological compositions.

The basis of MRI is magnetic resonance or nuclear magnetic resonance (NMR) spectroscopy. An NMR experiment is based on the resonant absorption of rf radiation by nuclei exposed to a magnetic field. “Nuclei,” as used herein, refers to atomic nuclei. “Atomic nuclei” refers to the positively charged central portions of atoms that comprise nearly all of the atomic mass and contain protons and neutrons except for hydrogen which contains one proton only. Many atomic nuclei possess spin angular momentum. A nucleus with a spin quantum number (I) has angular momentum and a magnetic moment. For example, I=1/2 for a proton. To be detectable by NMR and/or MRI, a nucleus must have a non-zero spin. An “MRI-detectable” sample therefore includes a compound having at least one nucleus with a non-zero spin I. MRI-detectable nuclei include, but are not limited to, ¹H, ¹³C, ¹⁹F, ³¹P, and ¹⁵N. The term, “MRI detectable chemical shift,” refers to a signal due to the absorption of rf radiation by one or more MRI-detectable nuclei. The signal leads to an NMR spectrum, which is a plot of absorption of rf radiation against chemical shift (δ).

For example, nuclear magnetic moments interact with a local magnetic field. The local field may differ from an applied field because the latter induces electronic angular momentum which gives rise to a small additional magnetic field δB at the nuclei.

δB=−δB

The additional field is proportional to the applied field. The ability of the applied field to induce an electronic current in the molecule and the strength of the resulting local magnetic field experienced by the nucleus, depend on the details of the electronic structure near the magnetic nucleus of interest, so nuclei in different chemical groups have different shielding constants (δ). Because the total local field is

B_{loc}=B+δB=(1−δ)B

The Larmor frequency is

$${υ}_{L} = \frac{{γ B}_{loc}}{2 π} = ( 1 - σ ) \frac{γ B}{2 π}$$

This frequency is different for nuclei in different environments. Hence different nuclei, even of the same element, come into resonance at different frequencies, e.g., when in different compounds. These resonant frequencies are used in the present invention to detect a compound of interest, e.g., ethanol, lactate, other metabolic products, and the like. It is conventional to express resonance frequencies in terms of chemical shift. The chemical shift is an empirical quantity which relates the difference between the resonant frequency (ν) of the nucleus in question and that of a reference standard, (ν^{o}).

$$δ = \frac{υ - {υ}^{°}}{{υ}^{°}} \times {10}^{6}$$

In general, the chemical shift is dependent on the electron density surrounding the nucleus in question. For example, ethanol is optionally detected in a sample by the presence of a characteristic chemical shift (δ) at 4.35, which is a triplet peak due to the resonance of the hydroxyl proton. For more information on chemical shifts of various types of molecules, see, e.g., Spectroscopic Identification of Organic Compounds, Fourth Edition, by Silverstein et al. (John Wiley and Sons, New York, 1981); and Organic Spectroscopy, by Brown et al.(John Wiley and Sons, New York, 1988).

When a magnetic resonance image is desired, e.g., of a brain or an artificially generated physical array, one resonance frequency or chemical shift is typically probed. For example, an MRI image of a brain images, e.g., the water content of the brain. Therefore, the rf pulse applied to excite the nuclei is applied at a frequency equal to the resonant frequency of the protons in a water molecule. All of the water protons resonate at the same frequency. However, imaging techniques apply a magnetic gradient to the sample, e.g., the brain or physical array, that causes the protons in water to resonate at different frequencies depending on their spatial location in the sample. The image therefore provides a map of the concentration of water in the sample.

In the present invention, the samples of the array are all simultaneously screened, e.g. for the presence of one or more particular compounds. For example, when screening for ethanol, all samples are probed at the resonant frequency of ethanol, which in conventional NMR is the same frequency for all samples. However, when imaging techniques are used to apply a magnetic gradient, each sample experiences a different magnetic field and so the ethanol protons resonate at different frequencies depending on their location in the array. The image produced provides an image of the concentration of ethanol in each sample compartment. This is described in more detail below.

Magnetic resonance is achieved by applying a magnetic field to the sample(s) to be studied and then applying a radio frequency pulse to the sample. In magnetic resonance imaging, the magnetic field is varied. A linear variation or field gradient applied across the sample means that atomic nuclei in different locations in the sample experience different magnetic field strengths (B₁ and B₂). Therefore, nuclei in different locations within the sample exhibit different resonance frequencies (ν₁ and ν₂). According to the Larmor equation:

ν=^{γB}/2π

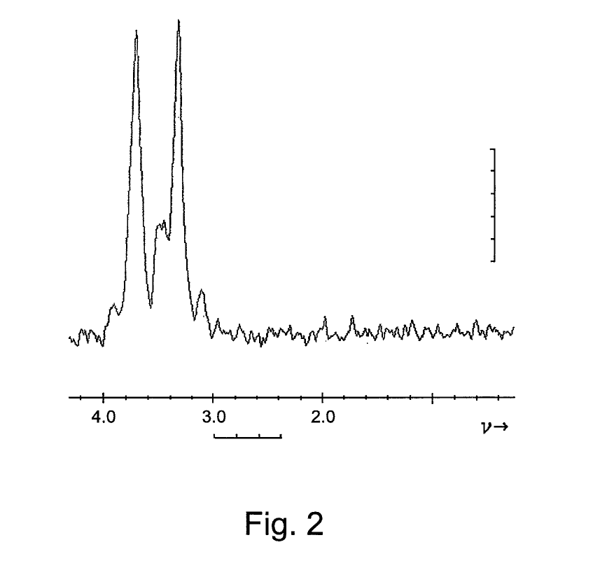

For a field gradient of 1×10⁻⁵ tesla/cm, ν=425 Hz. In other words, protons 1 cm apart along the field gradient in the sample have frequencies that differ by 425 Hz. Therefore, changing the probe pulse by 425 Hz, probes successive 1 cm positions in the sample. Each radio frequency (rf) pulse produces a free induction decay that encodes the concentration of the probed nucleic, e.g., protons, at each 1 cm position. By increasing the applied field gradient the thickness of the probed area decreases. In this manner, MRI imaging provides spatial information about the sample. The MRI image generated provides a convolution of an NMR spectrum, e.g., a proton signal, with a spatial concentration map. For example, the NMR signal provided in FIG. 2 shows an NMR spectrum of glucose. Convoluting the spectrum with a spatial concentration map obtained through MRI imaging provides a picture of an array of wells containing glucose. An image produced for this spectrum would contain a glucose signal for those wells containing glucose.

In the present invention, magnetic resonance techniques are applied simultaneously to a plurality of compounds positioned, e.g., in an artificially generated physical array. The samples within the physical array are placed in a magnetic field. Alternatively, a magnetic field is applied to the samples within the array. A radio frequency pulse or a continuous wave is applied to the samples, simultaneously exciting the nuclei at that resonant frequency in the various samples. For example, the resonant frequency of the hydroxyl proton of ethanol excites the hydroxyl proton, which then produces a signal which is detected. In continuous wave NMR the absorption signal is monitored as the frequency of the source is scanned. Alternatively, the frequency of the rf source is held constant while the strength of the magnetic field is scanned. In pulsed field magnetic resonance spectroscopy, the sample is irradiated with periodic pulses of rf energy that are directed through the sample at approximately right angles to the magnetic field. These excitation pulses elicit a time-domain signal, an FID, that decays in the interval between pulses. The FID is then converted to a frequency domain signal by using Fourier transformation to give a spectrum of resonant frequency or chemical shift. The intensity of the signals obtained in magnetic resonance is proportional to the number of nuclei present in the sample at the same resonant frequency.

In MRI, a field gradient is also applied to the sample to provide spatial information, as described above. The field gradient is optionally a one-dimensional or a two dimensional magnetic field gradient. A one-dimensional field gradient is a variation with respect to one dimension and a two-dimensional field gradient is a variation with respect to two directions. The typical symbols for a magnetic field gradient in the x, y, and z directions are Gₓ, G_{y} and G_{z}. For example, when a magnetic field gradient is applied to a physical array containing, e.g., 24 spatially separated sample compartments, each sample compartment experiences a different magnetic field, and therefore the protons of interest in that sample , e.g., the ethanol protons will exhibit different chemical shifts. The result is an NMR spectrum with more than one signal. The amplitude of the signal is proportional to the number of spins in a plane perpendicular to the gradient. This is called frequency encoding and is used to deconvolute the signal, thus providing the spatial location for each signal. Backprojecting is then optionally used to generate an image of the physical array.

Backprojecting is one of the simplest ways to provide an image in MRI. Other techniques are available, e.g., Fourier transform imaging, that are well known to those of skill in the art. In backprojecting, a one-dimensional field gradient is applied at several angles, and an NMR spectrum is recorded for each angle. Once recorded, the data can be deconvoluted or backprojected through space, e.g., using a computer. Fourier transform imaging is a more commonly used method of imaging in magnetic resonance. This technique involves a phase encoding gradient, a slice selection gradient, and a frequency encoding gradient. The three gradients are used to produce an FID for each spatial location screened. The FIDs are then fourier transformed to provide frequency and location information, e.g., for each sample well in a physical array.

MRI images are produced using, e.g., back projection imaging, fourier transform imaging, phase encoding gradients, slice selection gradients, frequency encoding gradients, and other techniques well known to those of skill in the art. In the present invention, differences in chemical compositions can be measured by 2D or 3D imaging of the distribution of characteristic chemical shifts using essentially the same techniques applied for metabolic imaging, e.g., of a brain. The characteristic chemical shifts in the present invention are chosen to correlate, e.g., to the presence and concentration of a particular composition. In addition, the samples are placed into an artificially generated array as described below. Methods for enhancing sensitivity and decreasing data acquisition time for MRI, e.g., for metabolic imaging of biological macro-objects are well known in the art. These methods are fully applicable to the methods provided herein.

One particularly useful technique for rapid data acquisition in MRI involves the use of spiral based k-space trajectories. Such a trajectory samples data in four-dimensional space, the four dimensions comprising kₓ, k_{y}, k_{z}, and k_{f}. The k-space dimensions constitute the fourier space that corresponds to the three spatial dimensions, x, y, and z, and one frequency or chemical shift dimension, f. Data is sampled along a k-space path based on the time integral of the gradient waveforms, in which the frequency coordinate is equal to time and the origin is defined as the echo time of the excitation sequence. During the readout period, the time-varying gradients trace out a spiral path in the kₓ, k_{y} plane. This technique is described in more detail in U.S. Pat. No. 5,652,516, by Adalsteinsson et al. In general, the method provides for rapid data acquisition with independent control over imaging time and spatial resolution. Data is scanned from three spatial dimensions in the same time with no loss of signal to noise ratio.

The spiral trajectories allow, e.g. for 3D metabolic mapping of a human brain using N-acetyl aspartate distribution with a nominal voxel resolution of 1.1 cm³, and with approximately 5000 data points for 3D image construction acquired in about 17 minutes. Each “datapoint” is an NMR spectrum with suppression of solvent and fat signals. Concentrations as low as the millimolar range were detected using spiral trajectories. Many variations of the MRI scanning parameters, field excitation techniques, and signal analyses methods are known in the art and are applicable to the present methods to obtain images for artificially generated arrays, e.g., of biological compositions.

Other techniques of interest in, e.g., decreasing the data acquisition time, include, but are not limited to, volume imaging, fractional nex and echo imaging, fast spin-echo imaging, chemical shift imaging, visible bandwidth imaging, spatially localized spectroscopy, echo planar imaging, and magnetic transfer contrast. See, e.g., Szumowski, D. B. Plewes, “Separation of Lipid and Water MR Imaging Signals by Chopper Averaging in the Time Domain, “Radiology 165:247-250 (1987); F. Forzaneh, S. J. Riederer, N. J. Pelc, “Analysis of T2 Limitations and Off-Resonance Effects on Spatial Resolution and Artifacts in Echo-Planar Imaging,” Magn. Reson. Med. 14:123-139 (1990); H. Bruder, H. Fischer, H. E. Reinfelder, F. Schmitt, “Image Reconstruction for Echo Planar Imaging with Nonequidistant k-Space Sampling, Magn. Reson. Med. 23:311-323 (1992); and J. Eng, T. L. Ceckler, R. S. Balaban, “Quantitative ¹H Magnetization Transfer Imaging in Vivo,” Magn. Reson. Med. 17:304-314 (1991).

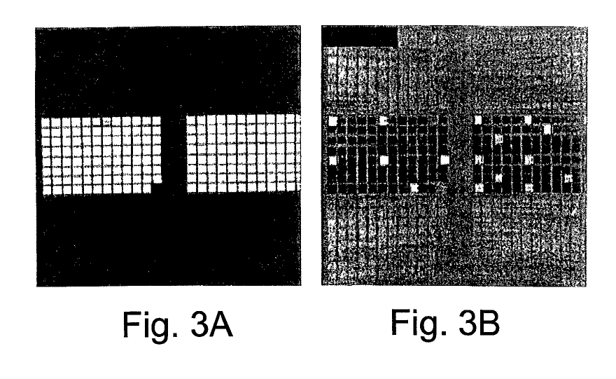

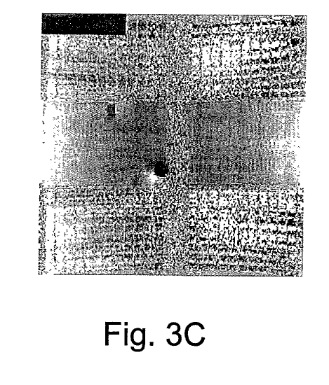

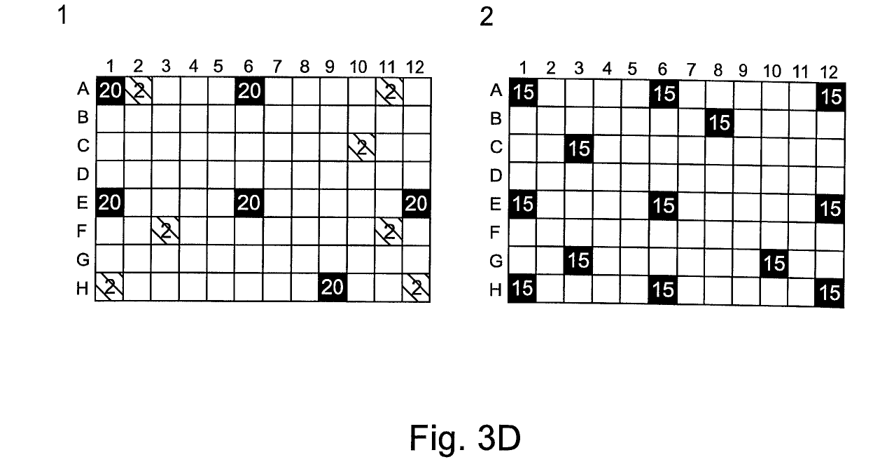

In the present invention, acquisition times typically range from about 1 minute to about 30 minutes. Preferably, acquisition times range from about 5 minutes to about 20 minutes. More typically, the time ranges from about 10 to about 15 minutes. For example, the spectrum in FIG. 2 of a 30 mM glucose solution was obtained from a sample comprising about 6 mls with a data acquisition time of about 2 minutes. Acquisition times for smaller samples, such as those used to obtain the images in FIG. 3, are typically a little longer. For example, a 20 minute turn around time is optionally obtained for a physical array comprising a plurality of 1.5-2.0 ml samples. The turn around time typically includes data acquisition as well as loading and removing the physical array from the spectrometer. Therefore, a physical array comprising 1536-well plates is optionally used to screen 55,296 samples in an hour.

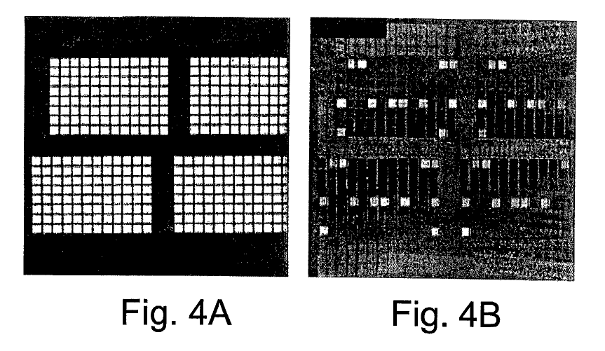

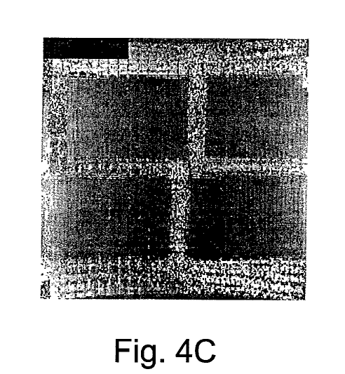

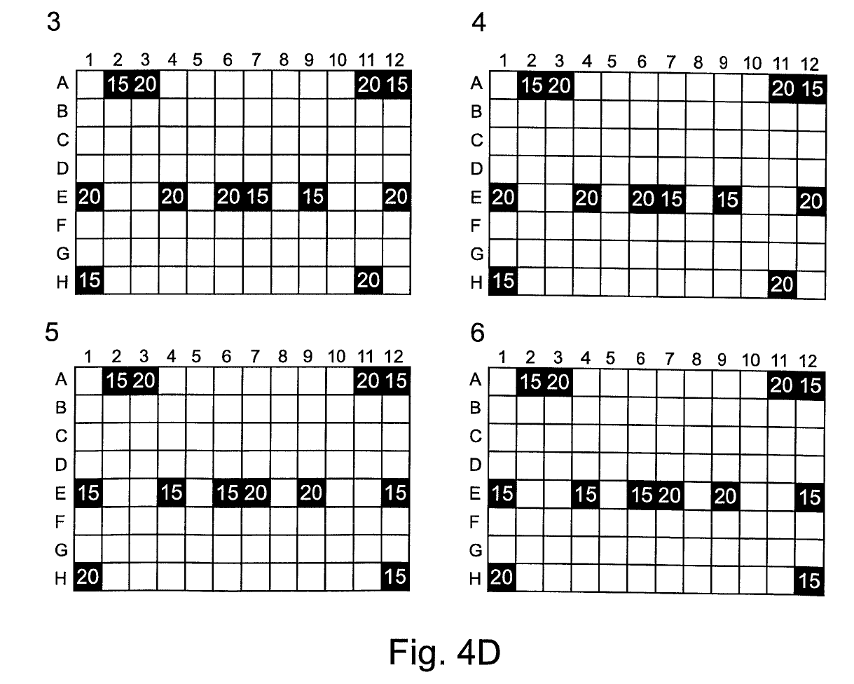

The images produced in traditional MRI provide a picture of a knee, back, head, or the like. In the present invention, the images produced provide a map of the physical array. For example, FIG. 4, Panel B shows a map of four 96-well microtiter plates. The horizontal and vertical lines correspond to the walls separating the various spatially separated sample compartments in a physical array of the present system. Each square corresponds to one sample compartment of the physical array and the intensity of the signal in each square corresponds to the amount of the compound of interest, ethanol in this example, detected in that sample compartment.

The images produced using the above methods are deconvoluted to provide information on the samples in the array. “Deconvoluting” refers to the process of correlating the images obtained from MRI with the various spatial locations in the array. Deconvolution in the present application comprises determining the position of each sample according to the physical map. Therefore, the deconvolution optionally involves providing a comparison between a marker sample compartment and the physical array. The deconvolution step provides an indication of which sample compartments comprise a signal, i.e., which sample compartments correspond to a sample containing the compound of interest. It further involves comparing the various signals and providing a signal level for each sample compartment and/or sample. It further involves quantifying the amount of a particular compound in a particular sample compartment by a determination of signal intensity in each sample compartment.

In the present invention, the images obtained are analyzed for the presence of a particular chemical shift corresponding to a compound of interest. For example, the images are optionally produced at a chemical shift indicative of ethanol, lactate, or both. An example of the type of image obtained is provided in FIGS. 3-6. FIGS. 3-6 provide images of multiple slices through an array of samples comprising twelve 96-well microtiter plates. Panel A in each figure provides an image of the water spectrum for each well in that layer of the array. Panel B provides an image of an ethanol spectrum for each well. Panel C provides a field map indicative of magnetic field homogeneity. For example, the two incongruities in Panel C of FIG. 3 (position 7 in the top row and position 12 in the bottom row) represent wells from which the sample leaked out during the experiment, thus causing the inhomogeneities. Panel D provides a key to what concentration of ethanol is in each well of the array. The images in these figures were obtained using 3 pt Dixon pulse sequence. See, e.g., Dixon, W. T., “Simple Proton Spectroscopic Imaging” Radiology, 153: 189-194 (1984).

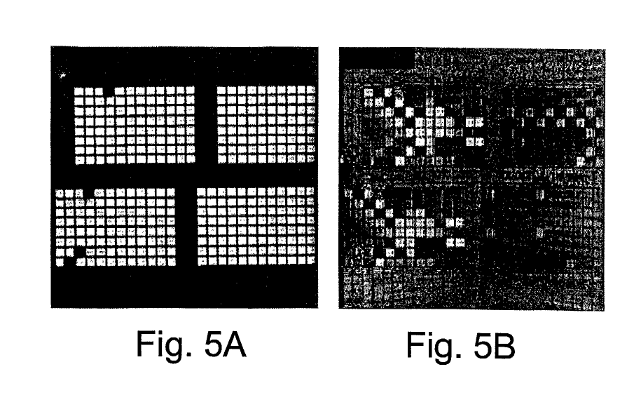

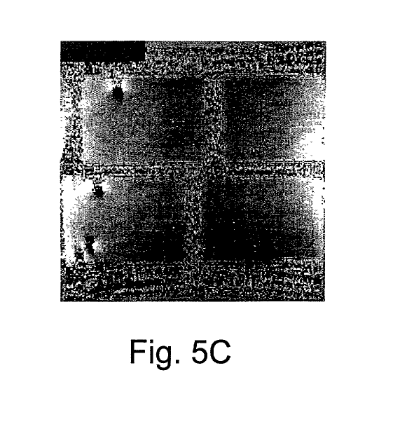

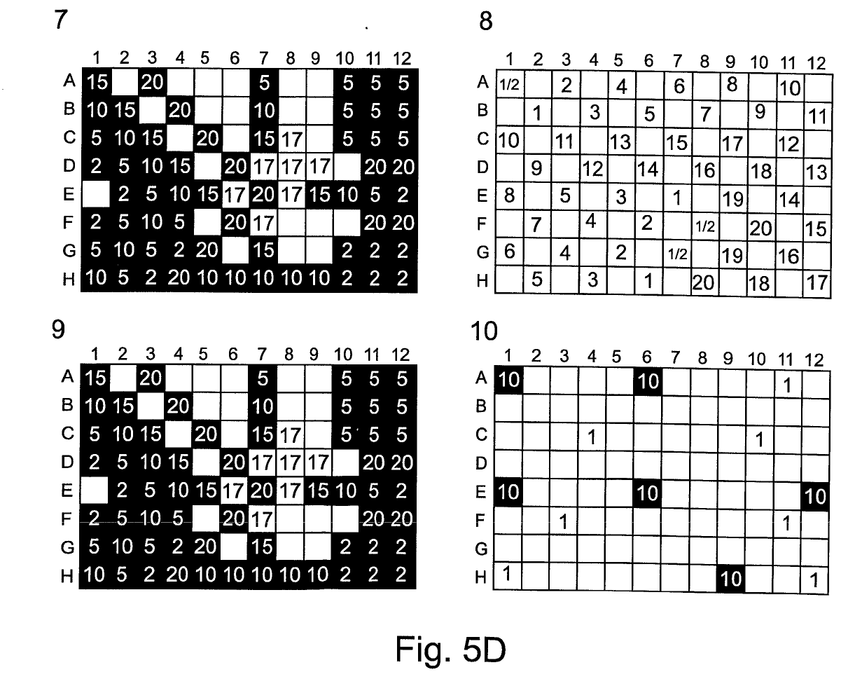

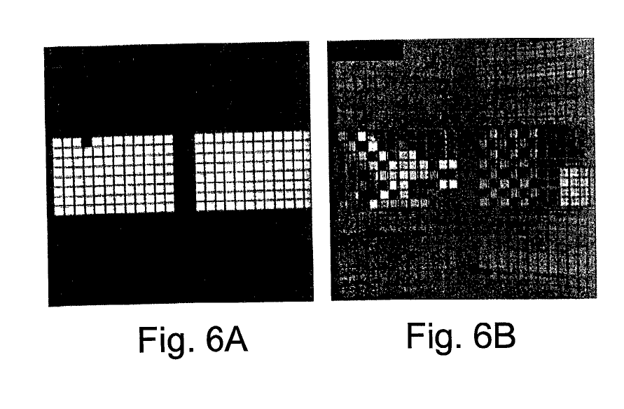

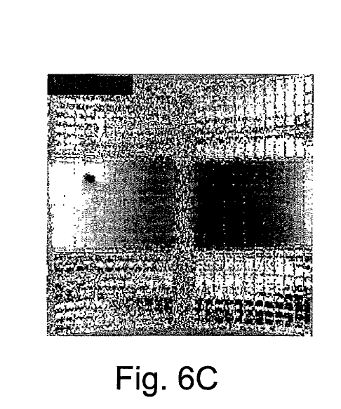

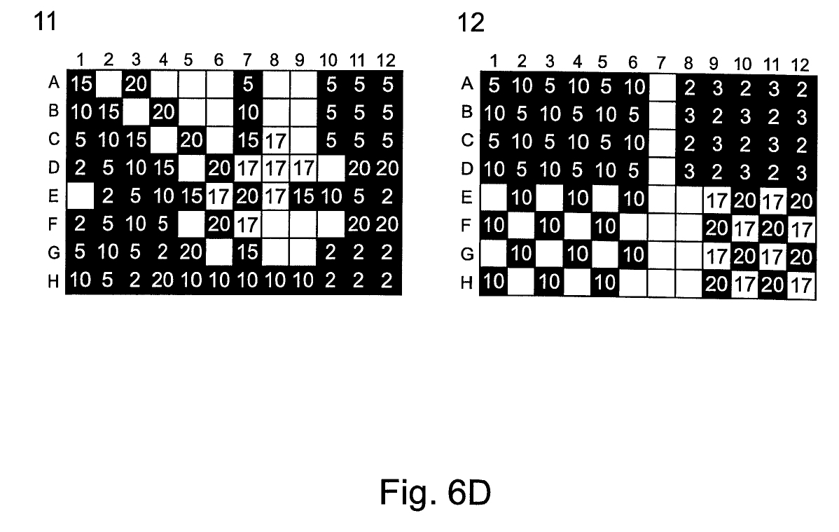

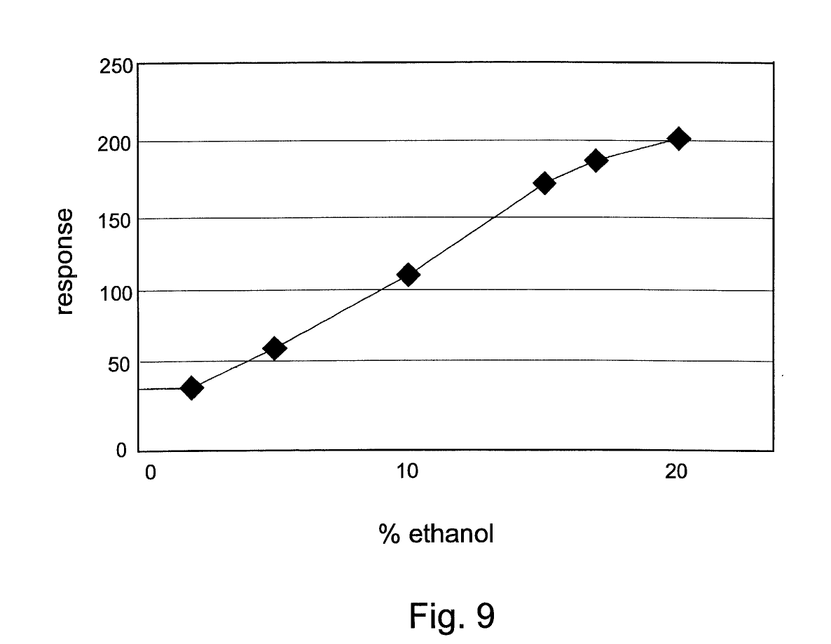

FIG. 3 shows the top two microwell plates of an array, which comprises multiple stacks of microwell plates. FIG. 4 shows four microwell plates in, e.g., the second level of the array. FIG. 5 shows a third level, comprising 4 microtiter plates and FIG. 6 shows the bottom level, comprising 2 microtiter plates. The sections of the images in which a signal appears correspond to the locations of samples containing ethanol. These wells are easily identified and checked against the key in Panel D. The intensity of the signals varies according to the concentration of ethanol in that particular sample. For example, in FIG. 6, the seventh column of the left-hand microwell plate in the map key in Panel D shows ethanol concentrations starting at 5% in the top row, going to 20% in row E and decreasing to 10% in row H. The corresponding image in Panel B shows corresponding increases and decreases in signal intensity along column seven. The concentration of ethanol corresponds to the signal intensity. FIG. 9 provides a quantitative representation, in terms of brightness vs. concentration, of ethanol samples from Plate 11, as depicted in FIG. 6.

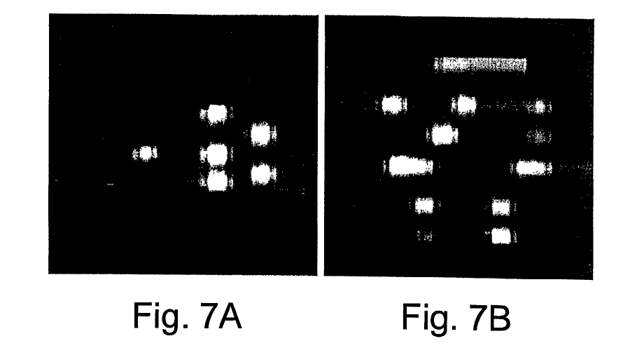

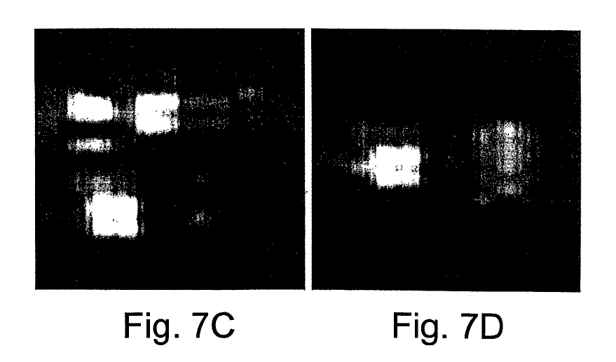

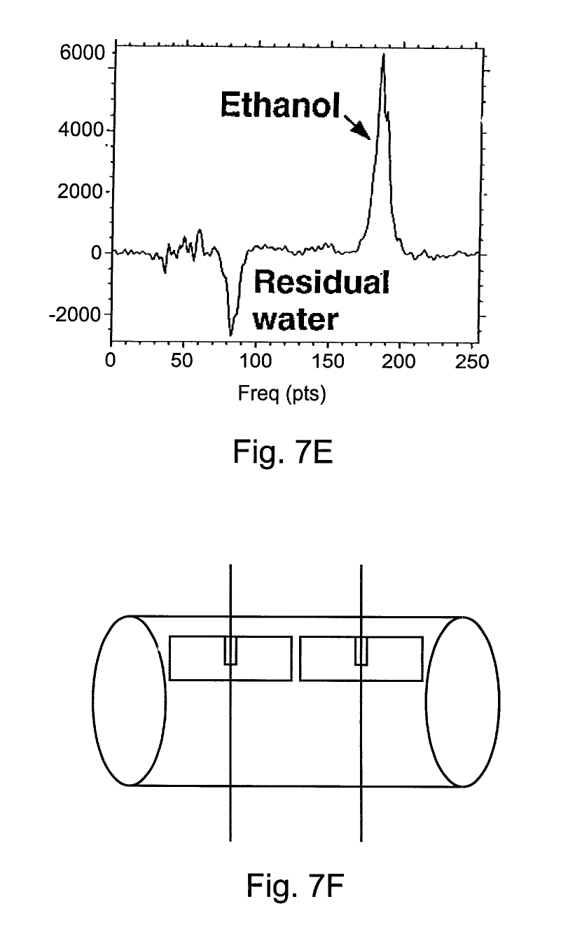

Alternative images of the same microwell plate array used in FIGS. 3-6 are provided in FIG. 7. These images were obtained using a volumetric spiral CSI” pulse sequence reformatted into coronal cuts. See, e.g., E. Adalsteinsson et al., “Volumetric Spectroscopic Imaging with Spiral-Based k-Space Trajectories” Magn Reson. Med. 39: 889-898 (1988).

For a review of MRI, see, e.g., Magnetic Resonance Imaging: Physical and Biological Principles, by Stewart C. Bushong, (Mosby-Year, 1995); MRI Principles, by Donald Mitchell, W B Saunders (1998); Magnetic Resonance Imaging: Mathematical Foundations and Applications, by W. Schempp ( Wiley-Liss 1998). For a review of magnetic resonance principles, see, e.g., Modern NMR Techniques for Chemistry Research, by Andrew Derome, (Pergamon Press 1987); and NMR in Physiology and Biomedicine, by Robert Gillies, (Academic Press, 1994). Many alternative methods for inducing magnetic resonance, e.g, using various pulse sequences, detecting the signals generated and producing images from the signals are readily apparent to those of skill in the art.

II. A High Throughput MRI Screening Apparatus

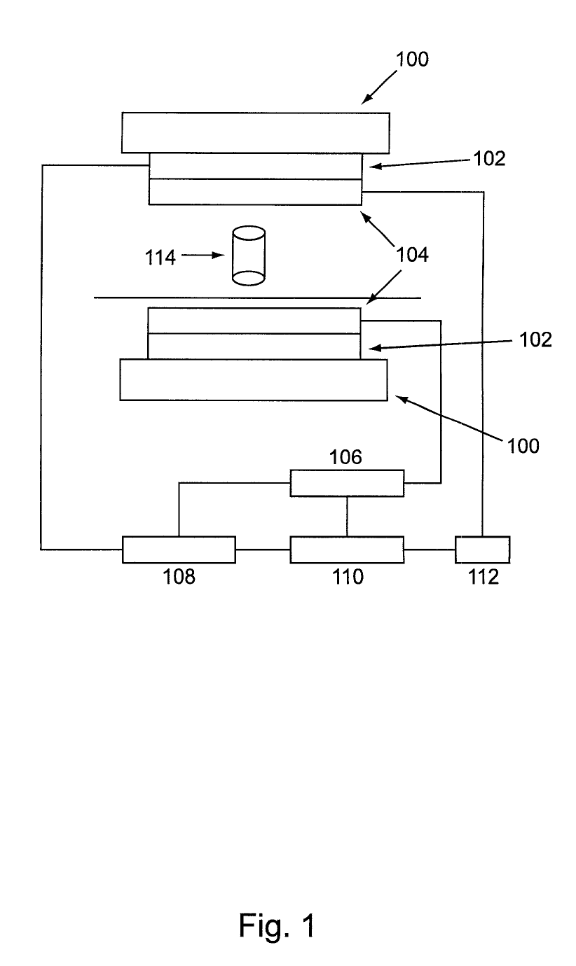

A magnetic resonance imaging spectrometer is typically used in the present invention to excite the nuclei of the samples, thus screening for a selected property. The spectrometer comprises a magnet, which magnet produces a magnetic field (B) used for imaging. The magnet used in an MRI spectrometer is typically a superconducting magnet, which typically produces a magnetic field strength of about 1 Tesla to about 12 Tesla. Preferably the field strength comprises about 1 Tesla to about 5 Tesla, more preferably about 1.5 Tesla to about 3 Tesla. The magnet also typically comprises gradient coils that produce a gradient in magnetic field B, e.g., in the x, y, and z directions. Within the gradient coils is an rf coil. The rf coil produces a B₁ field used to rotate the nuclei spins or net magnetization, e.g., by 45°, 90°, or 180°. The rf coil is also used to detect a signal from the spins. For example, the rf coils detect the transverse magnetization as it precesses in the xy plane. The detector used is typically a quadrature detector that separates out Mₓ and M_{y} signals from the rf coil. MRI spectrometers are available from a variety of manufacturers, e.g., GE, which makes the GE SIGNA 1.5 Tesla imager. A schematic of a spectrometer is shown in FIG. 1. Magnet 100, gradient coil 102, and rf coil 104 surround physical array 114, thus placing physical array 114 within the magnetic field produced by magnet 100. The physical array is also susceptible to the gradient and rf radiation applied across the magnetic filed. The physical arrays and methods of the present invention are optionally used with conventional MRI equipment using various data handling and representation software systems. Thus, the spectrometer, i.e., magnet 100, rf coil 104, and gradient coil 102, is typically coupled to computer 108, rf source 110, rf amplifier, 112, and rf detector 106.

A computer coupled to a spectrometer of the invention typically controls various components of the spectrometer. For example, the computer controls rf components, e.g., the radio frequency source, a pulse programmer, an amplifier, and the like. The rf source produces, e.g., a sine wave of the desired frequency, e.g., the resonant or Larmor frequency. A pulse programmer shapes the rf pulses, e.g., into apodized sine pulses. In addition, an rf amplifier is optionally used to increase pulse power, e.g., from milliwatts to kilowatts. The computer also optionally controls a gradient pulse programmer which sets the shape and amplitude of the gradient magnetic fields. An additional component is optionally an array processor, which is a device used to perform fast fourier transforms.

The computer, which optionally comprises an appropriately programmed processor, computer, or computer readable medium, is typically operably coupled to the MRI spectrometer and/or physical array and functions to instruct the operation of the apparatus of the invention, e.g., an MRI spectrometer, and its component elements in accordance with preprogrammed or user input instructions. The computer, processor, or other computer readable medium also optionally receives data and information from these instruments and interprets, manipulates, and reports this information to the user. As such, the computer is typically also appropriately coupled to one or more of: library storage elements, analog to digital or digital to analog control elements, and array processors, and the like.

The computer also typically includes appropriate software for receiving user instructions, e.g., in the form of user input into a set of parameter fields, e.g., in the form of preprogrammed instructions, e.g., preprogrammed for a variety of different specific operations. The software then converts these instructions to appropriate language for instructing movement of the physical array or control of the MRI spectrometer, and the like. The computer then receives data from one or more signal sensor/detectors included within the spectrometer system, and interprets the data, providing it in a user interpretable format, e.g., an image of each layer of the physical array, or using the data to initiate further instructions, such as in monitoring and control of sample rates, temperature, applied field and the like.

In the present invention, the computer typically includes software for acquiring magnetic resonance data, monitoring of materials in the MRI spectrometer, and deconvolution of the data to provide an image of the array. Additionally the software is optionally used to control insertion and placement of the physical array within the spectrometer.

Typically, a computer commonly used to transform signals from the detection device into, e.g., concentrations will be a PC-compatible computer (e.g., having a central processing unit (CPU) compatible with ×86 CPUs (e.g., a Pentium I, II or II class machine), and running an operating system such as, DOS™, OS/2 Warp™, WINDOWS/NT™, WINDOWS/NT™ workstation, or WINDOWS 98™), or a Macintosh™ (running MacOS™), or a UNIX workstation (e.g., a SUN™ workstation running a version of the Solaris™ operating system, a PC running LINUX, a PowerPC™ workstation or a mainframe computer), all of which are commercially common, and known to one of skill in the art. Data analysis software on the computer is then employed to deconvolute signal information. Software for these purposes is available, or can easily be constructed by one of skill using a standard programming language such as Visual Basic, Fortran, Basic, Java, or the like.

One of skill will immediately recognize that any, or all, of these components are optionally manufactured in separable modular units, and assembled to form an apparatus or system of the invention. Computers, rf coils, gradient coils, detectors, sample manipulation robots, and the like are optionally manufactured in a single unit, but more commonly are constructed as separate modules which are assembled to form an apparatus or system for analyzing a plurality of samples. Further, a computer does not have to be physically associated with the rest of the apparatus to be “operably linked” to the apparatus. A computer is operably linked when data is delivered from other components of the apparatus to the computer. One of skill will recognize that operable linkage can easily be achieved using either conductive cable coupled directly to the computer (e.g., USB, parallel, serial, ethernet, or phone line cables), or using data recorders which store data to computer readable media (typically magnetic or optical storage media such as computer disks and diskettes, CDs, magnetic tapes, but also optionally including physical media such as punch cards, vinyl media or the like) which is then accessed by the computer.

In traditional MRI, a patient is positioned within the magnetic field created by the magnet. The patient is typically placed on a table and positioned within a cavity within the magnet bore. For example, a patient is positioned on a computer controlled patient table that properly positions the patient within the magnetic field. In the present invention, the subject of the invention is a sample container or physical array containing samples to be screened, rather than a patient. The physical array is placed within the magnetic field or, alternatively, the magnetic field is applied to the plurality of samples. The arrays of the present invention are typically detectably coupled to, e.g., an MRI spectrometer during operation of the apparatus. “Detectably coupled” refers to placement of the array within the magnetic field of the spectrometer such that the samples within the array are excited and detected when performing MRI. Arrays useful in the present invention are described in more detail below.

An “automatic sampler” is a robotic handler that transports samples from one location to another. An automatic sampler is used, for example, to transport samples from a preparation plate into a physical array, e.g., a microwell plate or set of microwell plates. An automatic sampler is also used in the present invention to transport a physical array into an MRI spectrometer and properly position the array within the magnetic field during operation of the apparatus. Examples of automatic samplers include the Gilson 8-probe microtiter autosampler and the microtiter autosampler from CTC Analytics. Automatic samplers optionally include robotic handlers that are used to pick colonies, such as a Q-bot, and/or remove reagents to or from a preparation plate.

III. The Physical Array

A “physical array” refers to an artificially generated structure that comprises a plurality of spatially separated sample compartments. An “artificially generated array” refers to a non-naturally occurring structure comprising, e.g., multiple spatially separated objects, sample compartments, wells, or the like. Preferably, the artificially generated arrays of the invention do not correspond to a naturally occurring structure such as teeth, e.g., false teeth, or hair, e.g., a wig.

The sample compartments each optionally hold a different sample. For example, the sample compartments each optionally comprise a different genomic library member, a different randomly generated mutagenized cell, the metabolic products from various mutant cells, or the like.

The array optionally comprises one or more microwell or microtiter plates. For example, a microwell plate optionally comprises 96 different sample compartments or wells. Physical arrays of the invention made from one or more microwell plates, e.g., stacked microwell plates, comprise at least about 12 to about 10,000 spatial locations or wells. Such an array holds about 12 to about 10,000 samples, all of which are simultaneously screened using the methods of the invention. For example, a 24-well plate holds 24 samples; two 24-well plates hold 48 samples, three plates hold 72 samples, etc. Other microwell plates of use in the present invention include 96-well plates, 384-well plates, and 1536-well plates, and the like. Such plates or groups of plates optionally comprise about 24, 48, 72, 96, 192, 288, 384, 768, 1152, 1536, 3072, 4068, or 6144 different spatial locations or sample compartments. Therefore an equal number of samples are optionally screened in one MRI experiment.

The arrays are typically uniform arrays, such as a microwell plate. For example, each sample compartment or well has a standard size, shape and geometry. The standard size sample compartment typically holds a volume of about 1 μl to about 20 ml. Preferably, the sample volume is about 100 μl to about 10 ml. More preferably, the sample volume ranges from about 1.5 ml to about 2.0 ml.

The physical arrays of the present invention are optionally two-dimensional or three-dimensional arrays. Two-dimensional arrays are those arrays that lie within one plane, e.g., a single microwell plate in which all of the samples lie in the one plane, e.g., a single xy plane. Three-dimensional arrays are those that lie within two or more planes, e.g., multiple microwell plates. For example, a stack of microwell plates comprises a three-dimensional array. The stack has a plurality of samples that lie in multiple xy planes, e.g., along a z axis. A typical array of the invention comprises one or more stacks of microwell plates. Alternatively, the array comprises one or more racks of test tubes, or any other rack, plate or the like comprising multiples sample compartments.

In addition, the physical arrays of the invention optionally comprise a variety of shapes. For example, the arrays are optionally cylindrical arrays, square arrays, cubical arrays, rectangular arrays, or the like. The array is not necessarily limited to the geometry of a microwell plate. Any array containing spatially separated sample compartments is optionally used. For example a cylindrical array of diameter 20 cm and length 24 cm is optionally used to a house a plurality of sample compartments and, e.g., a plurality of samples. Alternatively, the array, e.g., a stack of microwell plates, is optionally placed into a separate structure for placement in the magnetic field. For example, a cylindrical structure of diameter 20 cm and length 24 cm is optionally used. Other structures include rectangular structures, cubical structures, and the like.

Typically, the structure and/or the array is configured from a non-metallic material, e.g., plastic, glass, polymers, and the like. The structure or the array itself optionally comprises paraffin filled outer walls. For example empty spaces between the wells are optionally filled with paraffin or water, e.g., doped water, e.g., water containing small amounts of MnCl₂, and the outer walls of the array are also optionally filled with or surrounded by, e.g., paraffin, water, or doped water. In fact, the entire array or structure is optionally surrounded by water when performing MRI spectroscopy.

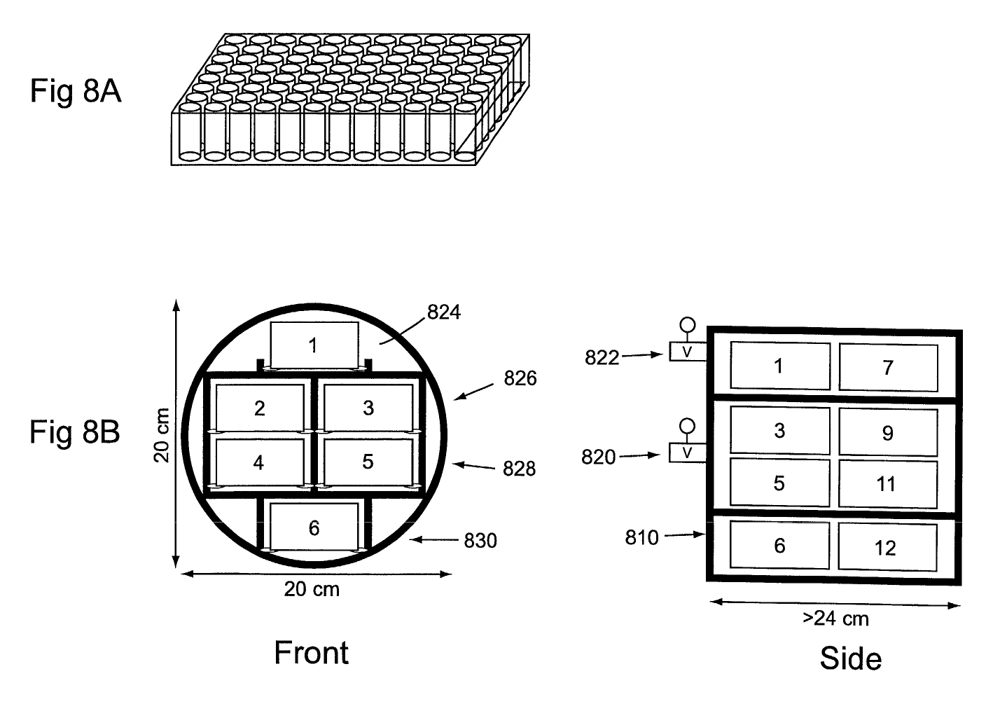

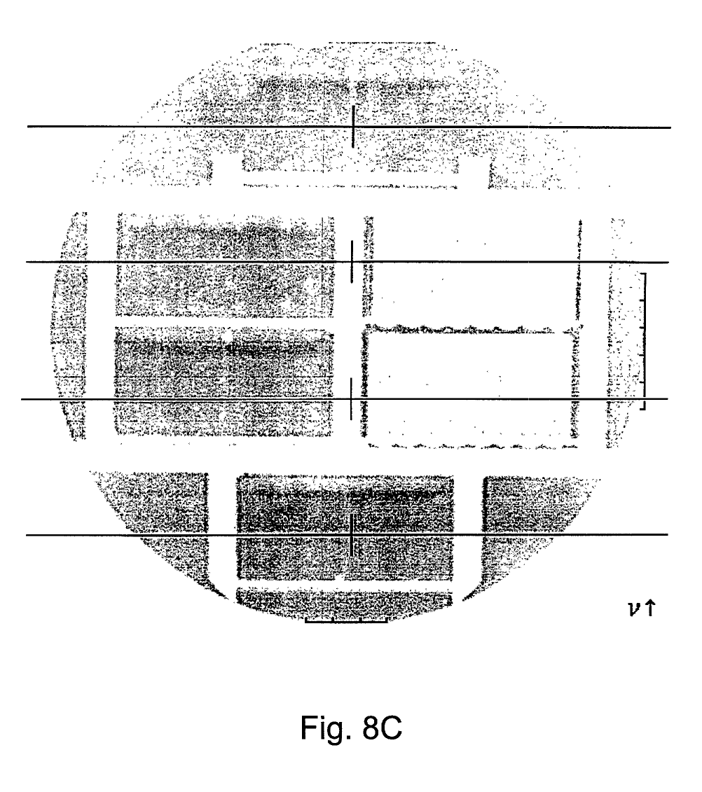

For example an image of an array of the invention is shown in FIG. 8, Panel C. A schematic of the array, e.g., comprising twelve microwell plates, is provided in Panel B of FIG. 8. The array optionally holds twelve 96-well microtiter plates, 1-12. For example, this array optionally holds twelve 96-well plates, i.e., up to 1152 samples. Alternatively, the array comprises multiple 384-well microtiter plates, 1536-well plates or the like. Test tube racks or any other artificially generated or manmade structure for holding multiple samples is optionally used in place of the array pictured. In the array pictured in FIG. 8, two plates are contained on top level 824, 4 on each of middle levels 826 and 828, and 2 on the bottom level of the array, level 830. The samples or plates are loaded into the array through a detachable wall or lid, e.g., lid 810. The spaces, e.g., between the various microwell plates, are optionally filled, e.g., with paraffin or doped water, or the like, e.g., through valves 820 and/or 822. The array pictured in FIG. 8 is about 20 cm tall by about 20 cm wide. However, arrays of other sizes and shapes, e.g., comprising less than 2 or greater than 12 microwell plates, are optionally used.

The arrays are optionally coupled to the spectrometers described above or to any available MRI or NMR spectrometer to form an apparatus of the invention. The apparatus as described is then used to simultaneously screen a plurality of samples in a high throughput mode.

IV. The Samples

“Samples” in the present invention typically comprise a compound or composition of interest. Each sample in the plurality of samples to be screened typically comprises an MRI-detectable compound, i.e., a compound that has at least one MRI-detectable nucleus, such as ¹H, ¹³C, ¹⁵N, ³³S, ³¹P, ¹⁹F, and the like, as described above. In addition, the compounds are optionally labeled or used in conjunction with NMR-active labeled precursors or substrates, such as compounds labeled with or enriched in ¹³C or ¹⁵N. Furthermore, the compounds optionally comprise one or more chiral shift reagents or derivatizing reagents, e.g., lanthanides, Mosher's reagent, or the like, or chiral solvents. These reagents are optionally used, e.g., to determine enantiomeric purity of chiral substances. A chiral shift reagent typically forms a complex with the chiral substance(s) and induces large NMR chemical shifts to produce strongly shifted spectra for two complexed enantiomers. This allows enantiomeric compounds to be easily distinguished and quantified since the relative intensities of the spectra correspond to the ratio of enantiomers in the sample.

Compounds of interest in the present invention include, but are not limited to, alcohols, polyols, carboxylic acids, lactones, esters, polyhydroxyalkanoates, terpenoids, carotenoids, steroids, polyketides, lipids, triglycerides, aromatics, amino acids, alkenes, vitamins, halogenated organic compounds, benzene bioconversion products, toluene bioconversion products, ethylbenzene bioconversion products, xylene bioconversion products, monosaccharides, polysaccharides, and the like. Examples of such compounds include, but are not limited to, ethanol, lactate, citrate, tylosin, 1,3-propanediol, succinate, glycerol, itaconate, PHB/PHA, lysine, threonine, glycine, isoleucine, methionine, tryptophan, phenylalanine, tyrosine, valine, glutamate, aspartate, histidine, phytohaemagglutinin-A, phytohaemagglutinin-B, p-hydroxybenzoate, 3-hydroxybutyrate, N-methylaspartate, GABA, aspartame, δ-caprolactone, and the like.

The compound of interest in the present invention is often a component of a biological composition or a member of a library of biological compositions. Such biological compositions include, but are not limited to, microbial cell cultures, mammalian cell cultures, cell biomasses, culture broths, extracts, reaction mixtures, plant tissues, fruit samples, root samples, tuber samples, plant seeds, and the like. These compositions are optionally placed directly in the sample compartments of the physical array and screened directly for a compound of interest by performing MRI spectroscopy. Alternatively, a plurality of compositions is optionally purified, e.g., in an off-line parallel purification system. Examples of such systems have been developed by the inventors and their coworkers, e.g., for use in mass spectrometry systems, as in U.S. Ser. No. 60/119,766 filed Feb. 11, 1999 and U.S. Ser. No. 09/502,283 filed Feb. 11, 2000, both entitled, “HIGH THROUGHPUT MASS SPECTROMETRY,” which are incorporated herein by reference. The systems are optionally used for magnetic resonance samples in the same manner as described in Ser. No. 60/119766 and Ser. No. 09/502,283. In other embodiments, the components of the biological compositions of interest are optionally purified and/or separated prior to MRI screening.

In some embodiments of the present invention, high throughput screening methods involve providing a library containing a large number of potential chemical or biological catalysts. The catalysts are optionally involved in a number of reactions of industrial importance, for example, oxidations, reductions, additions, cycloadditions, eliminations, polymerizations, depolymerizations, isomerizations, cyclizations, and the like.

In other embodiments of the present invention the samples comprise a library, e.g., a library of transformed cells, a library of variant genes, a library of mutagenized cells, a library of chemical compounds, or the like. A “library” of compositions or compounds in the present invention is a large collection of samples, e.g., composed of proteins, expression products, genes, nucleic acids, cells, pharmacologically active compositions, e.g., drugs, small organic molecules, peptides, and the like. The library of compositions or compounds optionally comprise a collection of individual molecules, or heterogeneous mixtures of compounds, such as cellular extracts, media collected from cultured cells, and chemical reaction mixtures (including, but not limited to, reactants, catalysts, products, and byproducts).

The libraries of the present invention typically comprise mutant cells, e.g., yeast cells that have been transformed, e.g., with a library of variant genes such as metabolic disorder genes, e.g., a shuffled library. Alternatively, a library of mutated cells, e.g., derived from the same parent, is used to identify mutants for a specific gene. For example, a library of mutated cells is optionally produced by random mutagenesis. Randomly mutagenized cells are produced, e.g., using ethylmethyl sulfate (EMS), dimethyl sulfate (DMS), methylnitosoguanadine (NTG), ethylnitrosourea (ENU), methylnitrosourea (MNU), hydrogen peroxide, UV light, gamma radiation, or the like. Alternatively, the libraries of the present invention comprise a plurality of variant gene sequences, e.g., shuffled gene sequences. The transformed cells, mutants, genes, or the like are then optionally screened by MRI, e.g., for the presence or concentration of one or more particular metabolites. For example, MRI is used to identify a mutant for a specific gene from a collection of different mutant strains obtained from random mutagenesis. Shuffled gene libraries, e.g., based on a specific gene identified, are optionally reintroduced in the mutants and screened to identify improved genes.

A library of variant genes is optionally produced by DNA shuffling, random mutagenesis, transposon mutagenesis, combinatorial gene assembly, and the like. Many such techniques are well known to those of skill in the art. In addition, the library is optionally composed of the expression products of a plurality of, e.g., variant genes, e.g., from a shuffled gene library.

A number of publications by the inventors and their co-workers describe nucleic acid diversity generation to produce libraries of nucleic acids which are expressed and screened. As adapted to the present invention, these methods are used to make and express libraries, which are then screened by MRI.

A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids that encode proteins with or which confer desirable properties. Following diversification by one or more of the methods herein, or otherwise available to one of skill, any nucleic acids that are produced can be selected for a desired activity or property. This can include identifying any activity that can be detected, for example, in an automated or automatable format, e.g., high throughput MRI as described herein or any of the assays in the art. A variety of related (or even unrelated) properties can be evaluated, in serial or in parallel, at the discretion of the practitioner.

Descriptions of a variety of diversity generating procedures for generating modified nucleic acid sequences are found in the following publications and the references cited therein: Stemmer, et al. (1999) “Molecular breeding of viruses for targeting and other clinical properties” Tumor Targeting 4:1-4; Ness et al. (1999) “DNA Shuffling of subgenornic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Certain U.S. applications provide additional details regarding various diversity generating methods, including “SHUFFLING OF CODON ALTERED GENES” by Patten et al. filed Sep. 28, 1999, (U.S. Ser. No. 09/407,800); “EVOLUTION OF WHOLE CELLS AND ORGANISMS BY RECURSIVE SEQUENCE RECOMBINATION”, by del Cardayre et al. filed Jul. 15, 1998 (U.S. Ser. No. 09/166,188), and Jul. 15,1999 (U.S. Ser. No. 09/354,922); “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549).

In brief, several different general classes of sequence modification methods, such as mutation, recombination, etc. are applicable to the present invention and set forth, e.g., in the references above. Any of these methods are optionally used to create libraries that can be screened using high throughput MRI arrays as described herein

The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above. A library produced by recursive recombination is optionally screened by high throughput MRI as described herein.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination.”

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202); “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579).

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/ gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in “METHODS FOR MAKING CHARACTER STRINGS, POLYNUCLEOTIDES & POLYPEPTIDES HAVING DESIRED CHARACTERISTICS” by Selifonov et al. , filed Jan. 18, 2000, (PCT/US00/01202) “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579). Extensive details regarding in silico recombination methods are found in these applications.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “SINGLE-STRANDED NUCLEIC ACID TEMPLATE-MEDIATED RECOMBINATION AND NUCLEIC ACID FRAGMENT ISOLATION” by Affholter, U.S. Ser. No. 09/656,549, filed Sep. 6, 2000.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity, which is optionally screened for desired properties using the methods described herein, e.g., MRI. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “METHODS FOR GENERATING AND SCREENING NOVEL METABOLIC PATHWAYS,” and Thompson, et al. (1998) U.S. Pat. No. 5,824,485 METHODS FOR GENERATING AND SCREENING NOVEL METABOLIC PATHWAYS) and their use to identify protein activities of interest has been proposed (In addition to the references noted above, see, Short (1999) U.S. Pat. No. 5,958,672 “PROTEIN ACTIVITY SCREENING OF CLONES HAVING DNA FROM UNCULTIVATED MICROORGANISMS”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “PRODUCTION OF ENZYMES HAVING DESIRED ACTIVITIES BY MUTAGENESIS.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 5 IF to lambda repressor” J. Biol. Chem. 264:13355-60); and “Walk-Through Mutagenesis” (Crea, R; U.S. Pat. Nos. 5,830,650 and 5,798,208, and EP Patent 0527809 B 1.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides, either of which are optionally screened by the MRI methods described herein.

In general, making libraries includes the construction of recombinant nucleic acids and the expression of genes in transfected host cells. Molecular cloning techniques to achieve these ends are known in the art. A wide variety of cloning and in vitro amplification methods suitable for the construction of recombinant nucleic acids such as expression vectors are well-known to persons of skill. General texts which describe molecular biological techniques useful herein, including mutagenesis, include Berger and Kimmel, Guide to Molecular Cloning Techniques, Methods in Enzymology volume 152 Academic Press, Inc., San Diego, Calif. (Berger); Sambrook et al., Molecular Cloning - A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)).

Methods of transducing cells, including plant and animal cells, with nucleic acids as in library construction, are generally available, as are methods of expressing proteins encoded by such nucleic acids. In the present invention, the libraries typically comprise cells that have been transformed with a library of genes, e.g., potential metabolic disorder genes. The cells are screened, e.g., to detect one or more metabolic products, to detect a level of metal uptake, to detect genes involved in metabolic disorders, or the like. In addition to Berger, Ausubel and Sambrook, useful general references for culture of animal cells include Freshney (Culture of Animal Cells, a Manual of Basic Technique, third edition Wiley- Liss, N.Y. (1994)) and the references cited therein, Humason (Animal Tissue Techniques, fourth edition W. H. Freeman and Company (1979)) and Ricciardelli, et al., In Vitro Cell Dev. Biol. 25:1016-1024 (1989). References for plant cell cloning, culture and regeneration include Payne et al. (1992) Plant Cell and Tissue Culture in Liquid Systems John Wiley & Sons, Inc. New York, N.Y. (Payne); and Gamborg and Phillips (eds) (1995) Plant Cell, Tissue and Organ Culture; Fundamental Methods Springer Lab Manual, Springer-Verlag (Berlin Heidelberg New York) (Gamborg). A variety of cell culture media are described in Atlas and Parks (eds), The Handbook of Microbiological Media (1993) CRC Press, Boca Raton, Fla. (Atlas). Additional information for plant cell culture is found in available commercial literature such as the Life Science Research Cell Culture Catalogue (1998) from Sigma- Aldrich, Inc (St Louis, Mo.) (Sigma-LSRCCC) and, e.g., the Plant Culture Catalogue and supplement (1997) also from Sigma-Aldrich, Inc (St Louis, Mo.) (Sigma-PCCS).

Alternative libraries of the invention comprise chemical libraries or libraries of chemical compositions, such as a library of potential modulatory compounds. Essentially any chemical compound can be used as a potential modulator or library member in the screening assays of the invention, although most often compounds can be dissolved in aqueous solutions, organic (especially DMSO-based) solutions are also used. Alternatively, a library or group of solid phase compositions is screened or assayed. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland), and the like. In one preferred embodiment, high throughput screening methods involve providing a combinatorial library containing a large number of potential therapeutic compounds (potential modulator compounds). Such combinatorial chemical libraries are then screened in one or more assays to identify those library members (particular chemical species or subclasses) that display a desired characteristic property, such as modulators that alleviate, e.g., reduction of metabolite levels related to a metabolic disorder. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks. Preparation of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with -D- glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidylphosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like). Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

V. Screening for Selected Properties

One of many possible applications for the present high-throughput screening method using MRI spectroscopy is the screening of a library of compositions for a desired property, e.g., metal uptake, pH, presence of a selected compound, or the like. A “selected property” of the invention is any desirable property of a composition, e.g., a protein, nucleic acid, pharmaceutical, small organic molecule, and the like, that can be screened or detected by MRI spectroscopy.

Once a desired property is selected, one or more MRI detectable chemical shifts related to the property are selected. The chemical shift correlates to the frequency at which the protons of the pertinent compound resonate as described above. For example, the chemical shift for the hydroxy proton of ethanol is optionally selected. The chemical shift of a phenolic proton is optionally selected to determine the pH of each sample in the library. Since substantially all molecules have a unique NMR spectrum, substantially any molecule chosen as a marker of the desired property has, e.g., a unique proton or carbon chemical shift to serve as a marker of the presence of the compound.

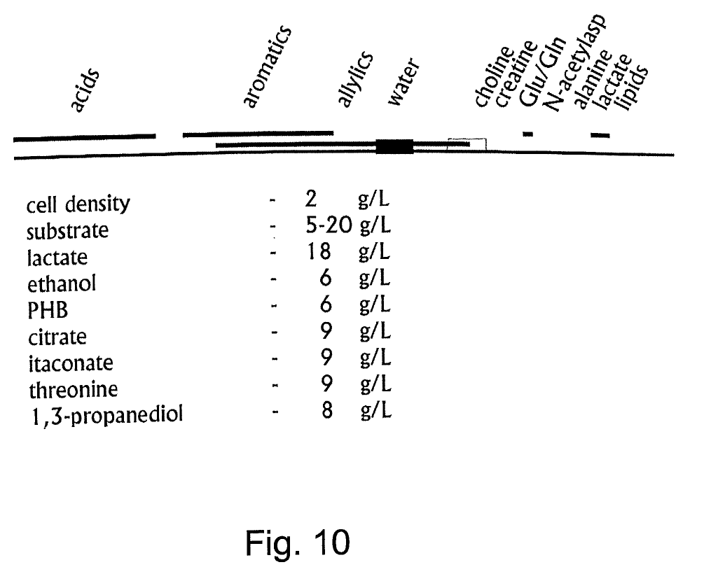

Further examples of desired or selected properties include, but are not limited to, metal uptake and concentration determinations. Metal uptake is optionally measured by including paramagnetic or ferromagnetic ions in the samples. The chemical shift monitored in this case varies depending on the metal ions used. Concentration of a compound of interest is optionally determined by choosing a chemical shift indicative of the compound and then measuring the presence and amplitude of the signal at that chemical shift for each of the samples in the array. FIG. 10 provides relative chemical shift positions for a variety of MRI-detectable moieties, e.g., acids, aromatics, allyic componds, water, choline, creatine, glutamic acid, glutamine, N-acetylaspartic acid, alanine, lactate, and lipids, and typical fermentation levels for a variety of compounds.

Compounds of interest include, but are not limited to, alcohols, polyols, carboxylic acids, lactones, esters, polyhydroxyalkanoates, terpenoids, carotenoids, steroids, polyketides, lipids, triglycerides, aromatics, amino acids, alkenes, vitamins, halogenated organic compounds, benzene bioconversion products, toluene bioconversion products, ethylbenzene bioconversion products, xylene bioconversion products, monosaccharides, polysaccharides, and the like.

Examples of such compounds include, but are not limited to, lactate, citrate, tylosin, 1,3-propanediol, succinate, glycerol, itaconate, PHB/PHA, lysine, threonine, isoleucine, methionine, tryptophan, phenylalanine, tyrosine, valine, glutamate, aspartate, histidine, phytohaemagglutinin-A, phytohaemagglutinin-B, p-hydroxybenzoate, 3-hydroxybutyrate, aspartame, δ-caprolactone, and the like.

Other applications include, e.g., the determination of expression levels, e.g., for a family of variant genes. For example, expression levels are optionally compared in a plurality of samples simultaneously by screening a plurality of samples, e.g., a plurality of transformed cells, at a chemical shift unique to one or more specific expression product.

When screening cells, the whole cells are optionally placed into the sample compartments of the array, such that metabolic products and/or gene expression levels in each cell are measured. For example, the content of a physical array optionally comprises 5000 individually grown microbial cultures. These cultures are then screened for one or more particular compound, produced, e.g., by means of fermentation or biotransformation. Alternatively, the cells are purified, e.g., using an off-line parallel purification system, and the expression products and/or metabolic products are placed into sample compartments after purification or separation.

MRI using the artificially generated arrays of the present system is particularly useful, e.g., for analyses of cell composition, including entire culture contents, or whole cell pellets and solvent extracts thereof. In addition, the arrays, e.g., of microbial cultures are optionally subjected to centrifugation prior to MRI analysis. In this instance, the analytes of interest are optionally measured in broth and in cell biomass from a single acquisition experiment. Thus, MRI using the arrays of the invention is also useful for parallel comparison of biocatalytic processes in various in-vivo fermentations, whole cell biotransformations, and in-vitro or cell free enzymes. Comparisons are optionally made of various growth rates, fitness, sustained productivity/compound excretion to the medium, and the like. These traits can all be tied to various sets of data on chemical shift distribution within the array of samples. For example, any compound of interest or class of compounds as mentioned above is available as a detected analyte for the above comparisons. For example, biomass content is measured to determine growth rates, and phosphate pools are optionally measured to determine fitness.

Alternative applications, include, but are not limited to, comparison of performance, e.g., of variants of biological compositions comprising, e.g., microbial strains expressing libraries of shuffled genes or gene clusters, microbial strain mutants or shufflants, microbial strains expressing genomic libraries, e.g., for cloning new genes with known function. This is applicable to samples including, but not limited to, plants, fruits, roots, tubers, whole seeds, and the like. For example, a library of seeds is optionally analyzed for essential oil content or chemical composition. Ratio of tissues/organs/pits, number and size of glands and glandular tissues are also optionally measured. Other examples include, but are not limited to, measuring the terpenoid content in wood or orange peel samples. Analysis of other compositions, such as poultry eggs is also available using the present methods.

In addition, the performance of a compound, gene, protein, drug, or the like, is optionally tested under a plurality of conditions simultaneously using MRI spectroscopy and the arrays of the present invention. For example, optimization of process conditions, establishment of growth requirements, and the like are easily performed in high throughput manner using the above methods for microorganisms, tissue samples, and the like. In addition, many more applications and embodiments of this technology will be readily apparent to those skilled in the art.

Once a desired property and an indicative chemical shift are selected, the plurality of compositions to be screened, e.g., a plurality of cells, e.g., transformed with a genomic library, is placed into the various sample compartments of a physical array, e.g., a microwell plate or stacked microwell plates, as described above. The physical array containing the plurality of samples to be screened is placed into a MRI spectrometer and MRI is performed on the entire library simultaneously. The samples are exposed to an rf pulse which comprises a frequency corresponding to the chemical shift related to the desired property. The nuclei in the samples are excited and detected as described above, thus producing an image of the physical array at each chemical shift of interest. The presence of a compound of interest is detected in the various sample compartments, e.g., corresponding to the various library members, by the presence of a signal in that location on the physical array image. The concentration of a compound of interest is optionally determined by the intensity of the signal in the corresponding position of the array.

Since an entire library of compounds, e.g., 10 to about 10,000 compounds is optionally positioned in a physical array for screening, the entire library is simultaneously screened in a high throughput manner by MRI methods provided herein. A screening rate of least about 3000 to about 50,000 samples per hour is optionally obtained. Preferably, the screening rate is about 5000 to about 50,000 samples per hour or about 10,000 to about 50,000 samples per hour. More preferably the sampling rate is at least about 20,000 to about 50,000 samples or more per hour.

VI. Identifying metabolic disorder genes

In an additional embodiment of the present invention, MRI screening is performed on a library of transformed cells or a library of mutated cells to identify genes that are involved with metabolic disorders, such as colon cancer, lupus nephritis, deafness distoria, Batten disease, and the like. Frequently, human disorders are caused by genetic aberrations that are not well understood on a molecular level and for which the metabolic defects are not clear. This is especially true when such defects result in reduced levels of metabolites as opposed to the absence of specific metabolites. The defect may even result in organ-localized metabolic changes that are hard to identify in blood composition analysis.

MRI is used in the present invention to identify mutations that cause reduced or increased levels of common metabolites. It has been shown repeatedly that mutations in S. cerevisiae can be linked to human genes that have been implicated in certain disorders. For examples of yeast-human disorder connections, see, e.g., Nature Genetics 1999, 22:55; PNAS 1999, 96:2141; PNAS 1999, 96:2104; Cell 1993, 75:1027. Since it has traditionally been difficult to identify S. cerevisiae mutants that produce reduced levels of, e.g., ethanol, lactate, specific sterols, fatty acids, or the like, relevant genes in metabolic disorders are still relatively unknown. MRI analysis, e.g., of yeast mutants, therefore provides identification of genes related to metabolic disorders by screening a library of cells or genes simultaneously for levels of one or more particular metabolic products. Using transposon mutagenesis, genes are optionally identified that affect the metabolic pathway of choice. In this way, even those mutations that do not cause a complete absence of the phenotype are optionally identified. In addition, this approach assures the identification of ancillary factors that are characterized by cloning of the insertion site and DNA sequencing.

Additionally, whole genome shuffling of such mutants is optionally used to address the relationship or genetic interdependence of individual genes. For instance, two regulatory genes in the same control pathway may in combination not reduce metabolite production, whereas such a pair of genes from different control pathways would reduce metabolite production.

Similar procedures are also optionally used to distinguish aberrant metabolic patterns in tissue cultures, plant cell cultures, germinating seeds, callus cultures, with and without hormone nutrient additions, hatching eggs, and developing embryos of fish, amphibia, and the like. For example, germinating seeds are optionally screened, e.g., for starch composition, fatty acids, and the like, and hatching eggs and developing embryos may be screened, e.g., for lactate, creatine, N-acetylaspartate, glycogen, fats, and the like.

As used herein, the term “metabolic disorder genes” refers to genes or gene sequences that play a role in causing, sustaining, or alleviating metabolic disorders. Metabolic disorders are disorders related to metabolism which refers to the processes, e.g., by which cells extract and store energy and synthesize the building blocks of macromolecules. Such disorders include, but are not limited to, colon cancer, Batten disease, lupus nephritis, and deafness distoria. Genes related to these disorders are identified by MRI screening according to the methods of the present invention.

For example, transformed cells or the metabolic products therefrom are provided for the analysis. Metabolites or metabolic products are compounds or compositions that are produced by cellular metabolism or processes. For example, ethanol is a metabolite associated with, e.g., alcoholism, hepatocellular carcinoma and alcoholic liver disease. Therefore ethanol is optionally detected, e.g., in a library of mutants, by the methods described herein to provide an indication of the effect, e.g., of various genetic mutations on alcoholism, hepatocellular carcinoma or alcoholic liver disease. Lactate is likewise associated with respiratory chain disorders, sensineural hearing loss, mitochondrial myopathy, hemiplegic migraine, and cardiomyopathy. Glutamate, N-methylaspartate, and GABA are metabolites associated with, e.g., schizophrenia. N-methyl aspartate is also associated with epilepsy and multiple sclerosis. And glycine is associated with, e.g., hypoglycemia. These metabolites are used as described herein to study the associated diseases, e.g., to determine the effect a particular mutation has on the disease.

A colony of cells, e.g., mutated cells or cells that have been transformed with a library of genes, e.g., related genes, mutant genes, shuffled genes, or the like, are grown and then screened to detect differences in metabolite production. Various genes, e.g., a library of shuffled genes are transformed, e.g., using transposon mutagenesis, into yeast cells, e.g., S. cerevisiae and then screened. Other cells of use in this procedure include, but are not limited to, a yeast cell, a bacterial cell, a plant cell, a tissue culture, a callus culture, an insect cell, a germinating seed, a hatching egg cell, a developing embryo cell, and the like. Alternatively a library of mutants, such as randomly mutagenized cells, is screened to identify particular genes, e.g., genes associated with metabolic disorders. A metabolic disorder gene is thus optionally identified from a library of shuffled genes or from a library of mutated cells using MRI.

Mutated cells and shuffled libraries are optionally used together to identify and improve genes associated with metabolic disorders. For example, a library of mutated genes is optionally screened to identify a metabolic disorder gene. The identified gene is then optionally used to create a shuffled library which is reintroduced into the host and screened by MRI to identify genes with improved functioning.

Cells that contain genes related to the disease in question will typically exhibit reduced, or increased levels of metabolites as compared to standard or non-mutant cells. For example, a normal, uninfected cell produces a certain level of a particular metabolite. This level is optionally used as a comparison to the level produced by a family or library of mutant genes. Alternatively, cells with mutant versions of genes involved in the disorder in question will exhibit an absence of a particular metabolite.

To screen the cells for metabolite levels, a particular compound or metabolite is selected and a particular NMR chemical shift indicative of that compound is selected. Alternatively, more than one metabolite and more than one chemical shift is selected. In that case, frequencies corresponding to all chemical shifts of interest are excited and imaged. An image of the physical array is optionally produced for each chemical shift of interest.

The cells are placed into the sample compartments of a physical array of the invention, e.g., into the wells of one or more microwell plate, and placed within a magnetic field of an MRI spectrometer. MRI is performed as described above and in many references well known to those of skill in the art.

The images produced by MRI provide a map of the physical array corresponding to the chemical shifts selected. For example, if ethanol is the metabolite of interest, then a chemical shift corresponding to one or more ethanol protons will be imaged. For example, FIGS. 3 through 6 provide maps of a physical array comprising 12 microwell plates. Each square in the images corresponds to a sample compartment or well in one of the stacked microwell plates. For example, in Panel B of each figure, each square comprising a signal corresponds to a well in the array that contains ethanol. The intensity of the signal is proportional to the amount or concentration of ethanol in the corresponding well of the array. This is further illustrated by a comparison of Panel B with Panel D in each of FIGS. 3-6.

The metabolite production for each sample, i.e. each sample compartment, will be compared to a standard or normal cell as described above. Those cells with altered metabolite production, e.g., reduced or increased production of, e.g., ethanol, are identified as cells containing metabolic disorder genes. The genes are then characterized by cloning and sequencing.

VII. Identifying modulatory compounds

In another embodiment, metabolic disorder genes, identified and characterized as described above, are optionally used to identify molecules that alleviate the observed metabolic symptoms as a step in the development of new drugs to treat the metabolic disorder at issue. The metabolic disorder genes are screened and identified as described above.

Cells that have been transformed with metabolic disorder genes or mutated cells comprising mutation(s) in a metabolic disorder gene are optionally incubated with potential modulatory compounds. Potential modulatory compounds are compounds, compositions, or the like that are screened for an effect on the metabolite production in that cell. Potential modulatory compounds include, but are not limited to, peptides, proteins, metabolic products, carbohydrates, lipids, nucleic acids, nucleotides, oligonucleotides, small organic molecules, and combinations thereof. A “small organic molecule” is one that has a molecular weight less than about 2000 daltons, more typically less than about 1500 daltons. Libraries of chemical compositions or compounds as described above are optionally used in the present method as potential modulatory compounds. Examples of modulatory compounds include, but are not limited to, erythromycin and cholesterol. For example, a drug that fights colon cancer optionally returns metabolite production in a colon cancer cell to its normal or standard level, which equals that of a non-diseased cell. Therefore, cells are incubated with potential modulatory compounds and the cells are screened by MRI as described above. Levels of metabolites are obtained for an entire library of potential modulatory compounds in one experiment using the high throughput methods of the present invention. The compounds that return cellular metabolism to a normal level, thereby alleviating the reduced or increased level of metabolite(s) produced by the mutant metabolic disorder gene, are then identified and characterized for use in drugs that fight the diseases in question.

VIII. Comparative Analysis of Chemical and Biological Catalysts

An alternative application for the present high-throughput screening methods using MRI spectroscopy is the screening and/or optimization of a library of catalysts for a desired property. Catalysts are involved in a number of reactions of industrial importance, including, but not limited to, oxidations, reductions, additions, cycloadditions, eliminations, polymerizations, depolymerizations, isomerizations, cyclizations, hydrogenations, reductive alkylations and combinations thereof. A comparative analysis of one or more desired catalytic properties can be used to discover novel catalysts, and/or optimize the use of catalysts known in the art to generate more efficient catalysts and reaction conditions. Desired catalytic properties include, for example, production of a particular reaction product, improved product yield, selectivity of the reaction, catalyst stability during the reaction, sensitivity or resistance to catalyst poisoning, and the like.

In one embodiment of the present invention, methods of identifying a catalyst are provided. The catalyst is optionally a biological catalyst (for example, an enzyme or a ribozyme), or a chemical catalyst. Biological catalysts include, but are not limited to, enzymes or enzyme mixtures (purified or not purified); crystallized or otherwise pre-formulated enzyme preparations, cell-based cultures or spores (prokaryotic or eukaryotic; free or immobilized; living or dead; intact, permeabilized, or amalgamated), and the like. Chemical catalysts include, but are not limited to, metal catalysts (such as nickel, palladium, platinum and ruthenium), Raney-type catalysts, zeolite catalysts, metallocenes and other organometallic catalysts, FCC (fluid catalytic cracking) catalysts, oxychlorination catalysts, methyl chloride catalysts, methyl amine catalysts, melamine catalysts, guard bed catalysts (used to protect the bulk catalysts from fouling and/or poisoning), and the like. Commercial sources of chemical catalysts and catalytic reagents include Activated Metals & Chemicals, Inc. (www.amcpmc.com), Akzo Nobel Catalysts (www.akzonobel-catalysts.com), Criterion Catalysts (www.criterioncatalysts.com), and Zeolyst International (www.zeolvst.com).

The methods of identifying a catalyst include (i) providing a plurality of assay solutions, which plurality of assay solutions comprises at least one reactant; (ii) providing a plurality of catalysts (iii) combining the plurality of assay solutions and the plurality of catalysts; (iv) performing magnetic resonance (MRI) spectroscopy on the plurality of assay solutions, thereby detecting one or more products generated by an action of the catalyst on the at least one reactant; and (v) identifying one or more of the plurality of catalysts that alter the level of the at least one of the one or more products, thereby identifying a catalyst. At least one member of the plurality of catalysts is added to the plurality of assay solutions. Optionally, the plurality of catalysts are omitted from at least one assay solution, such that the assay solution can be used as a control assay solution. The plurality of catalysts optionally comprises cells, cellular extracts, or media collected from cell cultures. Alternatively, the plurality of catalysts comprises a library of synthesized or isolated compounds, such as a combinatorial library of compounds.

In another embodiment of the present invention, methods of optimizing a catalyzed reaction are provided. A plurality of assay solutions comprising the catalyst and at least one reactant is provided. The plurality of assay solutions optionally comprises a plurality of cells, such as collections of microbial cultures or tissue samples. Alternatively, the plurality of assay solutions comprises a collection of enzymes, or a collection of chemical catalysts. During the method, at least one reaction parameter is varied, and the product of the reaction is monitored by MRI. Reaction parameters that can be varied include, but are not limited to, catalyst concentrations; pH; solvents; assay solution composition; environmental factors; the length of time the reaction is performed; and methods or composition of reagents used to stop the reaction.

Other factors that can be varied within the assay solution composition include the ratio of water to organic solvent(s); the presence of organic and/or inorganic ions; the presence and pressure of oxygen or other gases; and the presence of catalyst activators or deactivators, or other chemical compounds (for example, plasticizers) that can otherwise influence the progression of the chemical reaction. Environmental factors that can be varied include temperature, pressure, electromagnetic radiation, ultrasound or other radiofrequencies, and the like. For reactions involving catalysis of polymers; the compositions of the co-monomers for polymer synthesis, or composition of polymers for polymer modification or depolymerization reactions, can be varied and/or optimized. In addition, the structure of the reactant is optionally varied. A library of compounds, or a plurality of reactants is optionally provided, the members of which comprise structural variants of one or more compounds for which a desired chemical conversion is sought (for example, one or more compound sets can be provided, where each set is represented by various olefins, carboxylic acids, alcohols, phenols, amines and the like, such as those used in catalyst specificity analysis or in combinatorial chemistry).

The method of optimizing can further include determining a “window of operation” of a catalyst. Determining this window is accomplished, for example, by analyzing the factors (i.e., reaction parameters) that limited or expanded the performance of the catalyst in the assay solution. This analysis provides sets of ranges of the different reaction parameters over suitable operation conditions for the given catalyst. Optionally, the conditions influencing performance of a given catalyst are further examined and optimized within such a window of operation, by iterative application of the methods of the present invention. Additional arrays of samples or assay solutions are optionally exposed to reaction parameters set within more narrowly defined boundaries than that of the preceding set of assay solutions, to determine optimal conditions within the previously established window of operation. Thus, the performance of a given catalyst under varying reaction conditions is optionally determined using the methods of the present invention, providing information regarding the efficiency of the catalyst and optimization of the catalyzed reaction.

The methods of the present invention are also optionally applied to evaluate the performance of a number of non-catalytic chemical compositions, including, but not limited to, absorbing polymers, coagulating polymers, and inorganic absorbents. Such polymers are useful, or potentially useful, for industrial and drinking water treatment, solvent purification, compound separation or partitioning (for example, by liquid-liquid extraction), solid-phase extraction, and chromatographic applications.

IX. Kits

The apparatuses described herein are optionally packaged to include many, if not all, of the necessary reagents, e.g., libraries and physical arrays, for performing the preferred function of screening by MRI, e.g., for identifying compounds of interest, metabolic disorder genes, potential modulatory compounds, and the like. Such kits also optionally include appropriate containers, arrays, and instructions for using the devices or integrated systems herein as well as necessary reagents, and in cases where reagents are not predisposed in elements of the device, with appropriate instructions for introducing the reagents into the library storage or preparation medium (e.g., a microwell plate or duplicate dish), physical array, or MRI spectrometer of the device. Such kits typically include a physical array and any necessary reagents, e.g., a library of mutant cells, predisposed in the wells or separately packaged. Generally, such reagents are provided in a stabilized form, so as to prevent degradation or other loss during prolonged storage, e.g., from leakage. A number of stabilizing processes are widely used for reagents that are to be stored, such as the inclusion of chemical stabilizers (i.e., enzymatic inhibitors, microcides/bacteriostats, anticoagulants), the physical stabilization of the material, e.g., through immobilization on a solid support, entrapment in a matrix (i.e., a gel), lyophilization, or the like.

The discussion above is generally applicable to the aspects and embodiments of the invention described above. Moreover, modifications can be made to the method and apparatus described herein without departing from the spirit and scope of the invention as claimed, and the invention can be put to a number of different uses that will be apparent upon review of the detailed description of the invention.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method of screening a plurality of samples for a selected property, the method comprising:
(i) providing an artificially generated physical array, which physical array comprises one or more samples at each of a plurality of spatial locations;
(ii) placing the physical array in a magnetic field or applying a magnetic field to the physical array;
(iii) performing magnetic resonance imaging spectroscopy (MRI) on the one or more samples of the physical array, thereby identifying the spatial location for each of the one or more samples having one or more MRI detectable chemical shifts, which one or more chemical shifts correspond to the selected property, thereby screening the plurality of samples for the selected property.

2. The method of claim 1, wherein each member of the plurality of samples comprises one or more atomic nuclei and the performing MRI comprises:
(a) exciting the one or more atomic nuclei, thereby producing a plurality of signals;
(b) detecting the plurality of signals;
(c) generating one or more images from the plurality of signals, which one or more images correspond to the plurality of spatial locations;
(d) analyzing the one or more images for the presence of one or more selected chemical shifts; which one or more selected chemical shifts correspond to the selected property; and,
(e) deconvoluting the one or more images and the one or more selected chemical shifts to provide the spatial location for each of the one or more samples having the selected property, thereby screening the plurality of samples for the selected property.

3. The method of claim 1, wherein the physical array comprises a uniform array.

4. The method of claim 1, further comprising positioning the one or more samples of the physical array within one plane.

5. The method of claim 1, further comprising positioning the one or more samples of the physical array within multiple planes.

6. The method of claim 1, wherein the physical array comprises a cylindrical array, a square array, a cubical array, or a rectangular array.

7. The method of claim 1, comprising positioning the physical array within a cylindrical structure, a square structure, a cubical structure, or a rectangular structure.

8. The method of claim 6, wherein the cylindrical array has a diameter of about 20 cm and a length of about 24 cm.

9. The method of claim 1, further comprising constructing the physical array to comprise one or more microwell plates.

10. The method of claim 1, comprising constructing the physical array to comprise paraffin-filled outer walls.

11. The method of claim 1, comprising surrounding the physical array with water.

12. The method of claim 9, wherein the one or more microwell plates comprise one or more 24-well plates, 96-well plates, 384-well plates, or 1536-well plates.

13. The method of claim 12, comprising providing at least about 24, about 48, about 72, about 96, about 192, about 288, about 384, about 768, about 1152, or about 1536 different spatial locations.

14. The method of claim 1, wherein the one or more samples comprise at least about 12 to about 10,000 samples.

15. The method of claim 1, wherein the one or more samples comprise at least about 24, about 48, about 72, about 96, about 192, about 288, about 384, about 768, about 1152, about 1536, about 3072, about 4068, or about 6144 samples.

16. The method of claim 1, providing the one or more samples to comprise a library of biological compositions.

17. The method of claim 16, wherein the library of biological compositions comprises a library of mutated cells.

18. The method of claim 16, wherein the library of biological compositions comprises one or more expression products.

19. The method of claim 18, further comprising expressing a library of variant genes to produce the one or more expression products.

20. The method of claim 19, further comprising generating the library of variant genes by DNA shuffling, random mutagenesis, or combinatorial gene assembly.

21. The method of claim 1, wherein the one or more samples comprise one or more chemical catalysts.

22. The method of claim 1, wherein the one or more samples comprise one or more of: a microbial cell culture, a cell biomass, a culture broth, an extract, a reaction mixture, a chemical catalyst mixture, a plant tissue sample, a fruit sample, a root sample, a tuber sample, and a plant seed.

23. The method of claim 1, wherein the one or more samples comprise one or more MRI-active compounds.

24. The method of claim 23, wherein the one or more MRI-active compounds comprise one or more of: ¹H, ¹³C, ¹⁵N, ³³S, 31P, and ¹⁹F.

25. The method of claim 1, wherein the one or more samples comprise one or more chiral shift reagents.

26. The method of claim 1, wherein the one or more samples comprise one or more paramagnetic or ferromagnetic ions and the desired characteristic is metal uptake.

27. The method of claim 1, wherein each sample has a standard volume, a standard size, and a standard geometry.

28. The method of claim 27, wherein the standard volume ranges from about 1 μl to about 20 ml.

29. The method of claim 28, wherein the standard volume ranges from about 100 μl to about 10 ml.

30. The method of claim 29, wherein the standard volume is about 1.5 ml to about 2.0 ml.

31. The method of claim 1, wherein the selected property is a selected pH and performing MRI comprises measuring a phenolic proton signal corresponding to each sample.

32. The method of claim 1, wherein the selected property is a selected amount of a compound of interest or the presence of a compound of interest in the one or more samples.

33. The method of claim 32, wherein the compound of interest is selected from: an alcohol, a polyol, a carboxylic acid, a lactone, an ester, a polyhydroxyalkanoate, a terpenoid, a carotenoid, a steroid, a polyketide, a lipid, a triglyceride, an aromatic, an amino acid, an alkene, a vitamin, a halogenated organic compound, a benzene bioconversion product, a toluene bioconversion product, an ethylbenzene bioconversion product, a xylene bioconversion product, a monosaccharide, and a polysaccharide.

34. The method of claim 32, wherein the compound of interest is selected from: lactate, citrate, tylosin, 1,3-propanediol, succinate, glycerol, itaconate, PHB/PHA, lysine, threonine, isoleucine, methionine, tryptophan, phenylalanine, tyrosine, valine, glutamate, aspartate, histidine, phytohaemagglutinin-A, phytohaemagglutinin-B, p-hydroxybenzoate, 3-hydroxybutyrate, aspartame, and δ-caprolactone.

35. The method of claim 1, wherein step (iii) comprises providing a comparison of the absolute or relative amount of a compound of interest in each sample.

36. The method of claim 1, wherein step (iii) comprises simultaneously measuring one or more selected chemical shifts for each of the one or more samples, which one or more selected chemical shifts correspond to the selected property.

37. The method of claim 1, wherein the plurality of samples comprises of one or more microbial strains expressing a library of shuffled genes, a genomic library, or a library of mutated cells and wherein performing MRI comprises comparing an expression level for each member of the library of shuffled genes or of the genomic library.

38. The method of claim 1, wherein step (iii) comprises comparing performance of a selected biological composition under two or more different conditions.

39. The method of claim 1, wherein step (iii) comprises applying spiral-based k-space trajectories.

40. The method of claim 1, wherein the magnetic field comprises a magnetic field strength of about 1.5 Tesla or more.

41. The method of claim 1, further comprising screening the plurality of samples at a rate of at least about 3000 samples per hour to about 50,000 samples per hour.

42. The method of claim 41, further comprising screening the plurality of samples at a rate of at least about 5000 samples to about 50,000 samples per hour.

43. The method of claim 41, further comprising screening the plurality of samples at a rate of at least about 10,000 samples to about 50,000 samples per hour.

44. The method of claim 41, further comprising screening the plurality of samples at a rate of at least about 20,000 samples to about 50,000 samples per hour.

45. An apparatus for screening a plurality of samples, the apparatus comprising:
(i) a magnetic resonance imaging spectrometer; and,
(ii) at least one microwell plate or other artificially generated physical array, wherein during operation of the apparatus the at least one microwell plate or other artificially generated physical array is positioned within a magnetic field produced by the magnetic resonance imaging spectrometer.

46. The apparatus of claim 45, wherein the magnetic field comprises a field strength of about 1.5 Tesla or more.

47. The apparatus of claim 45, wherein the at least one microwell plate or other artificially generated physical array comprises one or more 24-well plates, 96-well plates, 384-well plates, or 1536-well plates.

48. The apparatus of claim 45, wherein the at least one microwell plate or other artificially generated physical array comprises at least about 24, about 48, about 72, about 96, about 192, about 288, about 384, about 768, about 1152, about 1536, about 3072, about 4068, or about 6144 spatially separated samples.

49. The apparatus of claim 48, wherein the spatially separated samples each comprise a volume of about 1 μl to about 20 ml.

50. The apparatus of claim 49, wherein the spatially separated samples each comprise a volume of about 100 μl ml to about 10 ml.

51. The apparatus of claim 50, wherein the spatially separated samples each comprise a volume of about 1.5 ml to about 2.0 ml.

52. The apparatus of claim 45, wherein the at least one microwell plate or other artificially generated physical array comprises one or more spatially separated sample compartments, which spatially separated sample compartments are positioned within a single plane.

53. The apparatus of claim 45, wherein the at least one microwell plate or other artificially generated physical array comprises one or more spatially separated sample compartments, which spatially separated sample compartments are positioned within multiple planes.

54. The apparatus of claim 45, wherein the at least one microwell plate or other artificially generated physical array comprises a cylindrical array, a square array or a cubical array.

55. The apparatus of claim 45, wherein the at least one microwell plate or other artificially generated physical array is positioned within a cylindrical structure.

56. The apparatus of claim 55, wherein the cylindrical structure comprises a diameter of about 20 cm and a length of about 24 cm.

57. The apparatus of claim 45, wherein the at least one microwell plate or other artificially generated physical array has at least one outer wall, which outer wall is a paraffin-filled outer wall.

58. The apparatus of claim 45, wherein the at least one microwell plate or other artificially generated physical array is surrounded by water or filled with water.

59. The apparatus of claim 45, further comprising an automatic sampler operably coupled to the magnetic resonance imaging spectrometer, which automatic sampler positions the at least one microwell plate or other artificially generated physical array within the magnetic resonance imaging spectrometer.

60. The apparatus of claim 45, further comprising a detector operably coupled to the spectrometer, which detector detects signals generated by operation of the magnetic resonance imaging spectrometer.

61. The apparatus of claim 45, further comprising a computer and software operably coupled to the apparatus for recording and analyzing data from the magnetic resonance imaging spectrometer.

62. The apparatus of claim 45, wherein during operation, the apparatus screens the plurality of samples at a rate of at least about 3000 samples to about 50,000 samples per hour.

63. The apparatus of claim 62, wherein during operation, the apparatus screens the plurality of samples at a rate of at least about 5000 samples to about 50,000 samples per hour.

64. The apparatus of claim 62, wherein during operation, the apparatus screens the plurality of samples at a rate of at least about 10,000 samples to about 50,000 samples per hour.

65. The apparatus of claim 62, wherein during operation, the apparatus screens the plurality of samples at a rate of at least about 20,000 samples to about 50,000 samples per hour.

66. A method of identifying metabolic disorder genes, the method comprising:
(i) providing a plurality of cells, which cells comprise a library of mutated cells or have been transformed with a plasmid containing one or more members of a library of gene sequences, which cells produce one or more metabolites;
(ii) performing magnetic resonance imaging (MRI) spectroscopy on the plurality of cells or on the one or more metabolites, thereby detecting the one or more metabolites;
(iii) identifying one or more cells that produce a reduced or increased level of at least one of the one or more metabolites, as compared to a standard, thereby identifying one or more metabolic disorder genes.

67. The method of claim 66, wherein the metabolic disorder is colon cancer, batten disease, deafness distonia syndrome, or lupus nephritis

68. A method of identifying a modulatory compound, the method comprising:
(i) providing a plurality of cells, which cells comprises a library of mutated cells or have been transformed with a plasmid containing one or more members of a library of gene sequences, which cells produce one or more metabolites;
(ii) performing magnetic resonance (MRI) spectroscopy on the plurality of cells or on the one or more metabolites, thereby detecting the one or more metabolites;
(iii) identifying one or more cells that produce a reduced or increased level of at least one of the one or more metabolites, as compared to a standard;
(iv) screening a plurality of potential modulatory compounds by MRI spectroscopy for alleviation of the reduced or increased level of the at least one of the one or more metabolites;
(v) identifying one or more of the plurality of potential modulatory compounds that alleviate the reduced or increased level of the at least one of the one or more metabolites, thereby identifying a modulatory compound.

69. The method of claim 68, wherein step (iv) comprises:
incubating the plurality of cells with the plurality of potential modulatory compounds;
performing MRI spectroscopy on the plurality of cells or on the one or more metabolites, thereby detecting the one or more metabolites produced by the plurality of cells in the presence of the one or more potential modulatory compounds;
comparing the one or more metabolites produced by the plurality of cells in the presence of the one or more modulatory compounds with the one or more metabolites produced in step (i).

70. The method of claim 69, wherein comparing comprises quantifying the amount of the one or more metabolites produced in the presence of the one or more modulatory compounds.

71. The method of claim 68, wherein the one or more potential modulatory compounds comprise one or more of: a peptide, a protein, a metabolic product, a carbohydrate, a lipid, a nucleic acid, a nucleotide, an oligonucleotide, a small organic molecule, and combinations thereof.

72. The method of claim 66 or claim 68, wherein the plurality of cells comprises one or more of: a yeast cell, a bacterial cell, a plant cell, a tissue culture, a callus culture, an insect cell, a germinating seed, a hatching egg cell, and a developing embryo cell.

73. The method of claim 66 or claim 68, wherein the plurality of cells comprises a plurality of mutant Saccharomyces cerevisiae cells.

74. The method of claim 66 or claim 68, wherein the library of gene sequences comprises at least about 1000 or more, about 5000 or more, about 10,000 or more, or about 100,000 or more members.

75. The method of claim 66 or claim 68, further comprising generating the library of gene sequences by DNA shuffling, random mutagenesis, transposon mutagenesis, or combinatorial gene assembly.

76. The method of claim 66 or claim 68, wherein the library of gene sequences comprises a library of related gene sequences.

77. The method of claim 66 or claim 68, wherein the library of related gene sequences comprises one or more colon cancer genes, one or more batten disease genes, one or more deafness distoria genes, or one or more lupus nephritis genes.

78. The method of claim 66 or claim 68, wherein the one or more metabolites comprise at least one MRI-active compound.

79. The method of claim 66 or claim 68, wherein the one or more metabolites include one or more of: ethanol, lactate, citrate, tylosin, 1,3-propanediol, succinate, glycerol, itaconate, PHB/PHA, lysine, threonine, isoleucine, methionine, tryptophan, phenylalanine, tyrosine, valine, glutamate, aspartate, histidine, phytohaemagglutinin-A, phytohaemagglutinin-B, p-hydroxybenzoate, 3-hydroxybutyrate, aspartame, and δ-caprolactone.

80. The method of claim 66 or claim 68, wherein the one or more metabolites include one or more of: an alcohol, a polyol, a carboxylic acid, a lactone, an ester, a polyhydroxyalkanoate, a terpenoid, a carotenoid, a steroid, a sterol, a fatty acid, a polyketide, a lipid, a triglyceride, an aromatic, an amino acid, an alkene, a vitamin, a halogenated organic compound, a benzene bioconversion product, a toluene bioconversion product, an ethylbenzene bioconversion product, a xylene bioconversion product, a monosaccharide, and a polysaccharide.

81. The method of claim 66 or claim 68, wherein the plurality of cells comprises a plurality of mutant cells and the standard comprises a non-mutant cell.

82. The method of claim 66 or claim 68, the method further comprising quantifying an amount of the one or more metabolites produced by each member of the plurality of cells.

83. The method of claim 66 or claim 68, comprising simultaneously performing MRI spectroscopy on each member of the plurality of cells, thereby simultaneously detecting the one or more metabolites.

84. The method of claim 66 or claim 68, comprising simultaneously screening at least 3000 to about 50,000 library members in about 1 hour or less.

85. The method of claim 66 or claim 68, further comprising screening the library of gene sequences at a rate of at least about 3000 to about 50,000 members per hour.

86. The method of claim 66 or claim 68, further comprising screening the library of gene sequences at a rate of at least about 5000 to about 50,000 members per hour.

87. The method of claim 66 or claim 68, further comprising screening the library of gene sequences at a rate of at least about 10,000 to about 50,000 members per hour.

88. The method of claim 66 or claim 68, further comprising screening the library of gene sequences at a rate of at least about 20,000 to about 50,000 members per hour.

89. The method of claim 66 or claim 68, wherein performing MRI spectroscopy on the plurality of cells or screening a plurality of potential modulatory compounds by MRI spectroscopy comprises:
(a) providing an artificially generated physical array, which physical array comprises a plurality of spatial locations; wherein each member of the plurality of spatial locations comprises at least one member of the plurality of cells, which at least one member of the plurality of cells comprises at least one member of the library of gene sequences.
(b) placing the physical array in a magnetic field or applying a magnetic field to the physical array; and
(c) performing MRI spectroscopy on plurality of cells within the physical array, thereby identifying the spatial location for each of the one or more cells that produce a reduced or increased level of at least one of the one or more metabolites as compared to the standard.

90. The method of claim 89, wherein step (c) comprises:
(d) exciting one or more atomic nuclei in the one or more metabolites, thereby producing a plurality of signals;
(e) detecting the plurality of signals;
(f) generating one or more images from the plurality of signals, which one or more images correspond to the plurality of spatial locations;
(g) analyzing the one or more images for the presence of one or more selected chemical shifts; which one or more selected chemical shifts correspond to the one or more metabolites; and,
(h) deconvoluting the one or more images and the one or more selected chemical shifts to provide the spatial location for each of the one or more cells having the one or more metabolites.

91. The method of claim 89, wherein the physical array comprises a uniform array.

92. The method of claim 89, further comprising positioning the plurality of cells or the one or more metabolites within one plane in the physical array.

93. The method of claim 89, further comprising positioning the plurality of cells or the one or more metabolites within multiple planes in the physical array.

94. The method of claim 89, wherein the physical array comprises a cylindrical array, a square array, a cubical array, or a rectangular array.

95. The method of claim 89, comprising positioning the physical array within a cylindrical structure, a square structure, a cubical structure, or a rectangular structure.

96. The method of claim 95, wherein the cylindrical array has a diameter of about 20 cm and a length of about 24 cm.

97. The method of claim 89, further comprising constructing the physical array to comprise one or more microwell plates.

98. The method of claim 89, comprising constructing the physical array to comprise paraffin-filled outer walls.

99. The method of claim 89, comprising surrounding the physical array with water.

100. The method of claim 97, wherein the one or more microwell plates comprise one or more 24-well plates, 96-well plates, 384-well plates, or 1536-well plates.

101. The method of claim 100, comprising providing at least about 24, about 48, about 72, about 96, about 192, about 288, about 384, about 768, about 1152, or about 1536 different spatial locations.

102. The method of claim 89, wherein the plurality of cells comprises at least about 12 to about 50,000 samples.

103. The method of claim 89, wherein the plurality of cells comprises at least about 24, about 48, about 72, about 96, about 192, about 288, about 384, about 768, about 1152, about 1536, about 3072, about 4068, or about 6144 samples.

104. The method of claim 89, wherein step (c) comprises applying spiral-based k-space trajectories.

105. The method of claim 89, wherein the magnetic field comprises a magnetic field strength of about 1.5 Tesla or more.

106. The method of claim 89, further comprising screening at least about 3500 samples, about 5000 samples, about 10,000 samples, about 20,000, or about 50,000 samples per hour.

107. A method of identifying a catalyst, the method comprising:
(i) providing a plurality of assay solutions, which plurality of assay solutions comprises at least one reactant;
(ii) providing a plurality of catalysts;
(iii) combining the plurality of assay solutions and the plurality of catalysts;
(iv) performing magnetic resonance (MRI) spectroscopy on the plurality of assay solutions, thereby detecting one or more products generated by an action of one or more members of the plurality of catalysts on the at least one reactant; and
(v) identifying one or more members of the plurality of catalysts that alter the level of the at least one of the one or more products, thereby identifying a catalyst.

108. The method of claim 107, wherein the catalyst comprises a chemical catalyst or a biological catalyst.

109. The method of claim 107, wherein the plurality of catalysts comprises a plurality of cells.

110. The method of claim 107, wherein the plurality of catalysts comprises a library of compounds.

111. The method of claim 107, wherein the action of the catalyst on the at least one reactant comprises oxidation, reduction, addition, cycloaddition, elimination, polymerization, depolymerization, isomerization, cyclization, hydrogenation, reductive alkylation, or combinations thereof.

112. The method of claim 107, wherein identifying one or more of the plurality of potential chemical catalysts that alter the level of the at least one of the one or more products comprises analyzing product yields, compositions, reaction selectivity, catalyst stability, catalyst poisoning, or combinations thereof.

113. A method of optimizing a reaction condition for a catalyst, the method comprising:
(i) providing a plurality of assay solutions, which plurality of assay solutions comprises a reactant and a catalyst;
(ii) exposing the plurality of assay solutions to one or more of a plurality of reaction conditions;
(iii) performing magnetic resonance (MRI) spectroscopy on the plurality of assay solutions, thereby detecting one or more products generated by an action of the catalyst on the reactant;
(iv) identifying one or more of the plurality of reaction conditions that alter the level of at least one of the one or more products; and
(v) analyzing the one or more of the plurality of reaction conditions, thereby optimizing the reaction condition for the catalyst.

114. The method of claim 113, wherein the plurality of reaction conditions comprises catalyst concentration, catalyst activating agents, catalyst deactivating agents, solvent concentration, assay solution pH, pressure, temperature, electromagnetic radiation, reactant composition, length of reaction time, stop reagents, or combinations thereof.

115. The method of claim 113, wherein analyzing the one or more of the plurality of reaction conditions comprises determining a window of operation for the catalyst.
